(19) United States
(12) Patent Application Publication
(10) Pub. No.: US 2013/0244936 A1
(43) Pub. Date: Sep. 19, 2013

(54) CONSTITUTIVELY ACTIVE PROLACTIN RECEPTOR VARIANTS AS PROGNOSTIC MARKERS AND THERAPEUTIC TARGETS TO PREVENT PROGRESSION OF HORMONE-DEPENDENT CANCERS TOWARDS HORMONE-INDEPENDENCE

(71) Applicants: Vincent Goffin, Paris (FR); Sophie Bernichtein, Paris (FR); Isabelle Fernandez, Paris (FR); Philippe Touraine, Paris (FR)

(72) Inventors: Vincent Goffin, Paris (FR); Sophie Bernichtein, Paris (FR); Isabelle Fernandez, Paris (FR); Philippe Touraine, Paris (FR)

(21) Appl. No.: 13/701,916

(22) PCT Filed: Jun. 1, 2011
(86) PCT No.: PCT/EP2011/059124

§ 371 Date: Feb. 15, 2013

(30) Foreign Application Priority Data
Jun. 4, 2010 (EP) 10305597.6

(51) Int. Cl. C12Q 1/68

(52) U.S. Cl. CPC C12Q 1/6886
USPC 514/11.5; 435/6.12; 435/6.11; 506/9

(57) ABSTRACT

The present invention relates to an in vitro method of prognosing outcome of hormone-dependent cancer in a subject comprising detecting a constitutively activating mutation in the prolactin receptor (PRLR) gene in a nucleic acid sample previously obtained from said subject, said constitutively activating mutation being preferably a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine.

The present invention concerns the identification of a prognosis marker of tumoral progression of hormone-dependent cancers such as breast cancer and prostate cancer.

Hormone-dependent cancers, including prostate and breast cancers, are significant causes of morbidity and mortality in the Western world.

Breast cancer, which is the most frequent female cancer, still represents a major public health issue despite the huge progress made in term of prevention and therapeutic success. Indeed, it is assessed that one in nine women will develop a breast cancer during her life. This represents in France 41,000 newly diagnosed cases a year and 11,000 deaths (Rochefort and Rouesse (2008) Bul. Acad. Natl. Med. 192:161-179).

Breast cancers are very different depending in particular on the evolution stage and on the location and the cells from which they spread. Breast cancer is thus a very heterogeneous disease, gathering a high number of biological entities associated with specific pathological characteristics and different clinical behavior. Genomic analyses enabled to discriminate the following 5 types of breast cancer, depending on the expression of specific proteins, in particular of estrogen receptors and HER2: normal-like type breast cancer, “luminal” A type breast cancer (representing between 25 and 40% of the cases), “luminal” B type breast cancer (representing from 20 to 25% of the cases), basal-like type breast cancer (representing 15 to 18% of the cases) and HER2+ type breast cancer (representing about 15% of the cases) (Institut Curie (2010) Press kit: “Cancers du sein. A I'Institut Curie, ('innovation au service des femmes”).

Basal-like type breast cancers are characterized among others by the absence of estrogen receptors and progesterone receptors and by the fact that they do not over-express HER2. It is to be noted that cancer wherein HER2 receptor is not over-expressed (i.e. absence of HER2 receptor (HER2 is not detectable, in particular by immunohistochemistry), or HER2 receptor is expressed either normally or is under-expressed) is defined as “HER2 negative”, by opposition to cancer wherein HER2 receptor is over-expressed (due to amplification of HER2 gene) which is defined as “HER2 positive”. Basal-like type breast cancers are thus often assimilated to triple negative breast cancers. Whereas every basal-like type breast cancer is a triple negative breast cancer, some triple negative breast cancers do not belong to the basal-like type.

Hormone-independent breast cancers, including hormone receptor negative breast cancers (i.e. cancer characterized by the absence of estrogen receptors and progesterone receptors) and triple negative breast cancer, are still badly understood and their increased aggressiveness makes their treatment problematic. Indeed, the loss of hormonal receptors does not enable the patients to benefit from targeted treatments such as Tamoxifen (targeting estrogen receptor), or for triple negative only, Herceptin (targeting HER2). Additionally, these cancers are often more resistant to chemotherapies, leading to an often inevitably metastatic recurrence and finally death.

Accordingly, there is a need for methods enabling prognosis of the progression of a hormone-responsive breast cancer towards a hormone non responsive breast cancer, including hormone receptor negative and triple-negative breast cancer, in order to administrate to the patient a potentially effective treatment as early as possible

Prolactin (PRL) is a polypeptide hormone mainly secreted by lactotrope cells of anterior pituitary. PRL is notably involved in mammary gland development and has a prominent role in lactation (Stricker and Grueter (1928). C. R. Soc. Biol. 99:1978-198). More than 300 separate functions or molecules activated by its receptor (prolactin receptor or PRLR), which belongs to the cytokine receptor superfamily, have been reported (Bole-Feysot et al (1998) Endocr. Rev. 19:225-268). Like most of the cytokine receptors, PRLR activates the JAK/STAT signaling pathway. Briefly, binding of PRL is assumed to induced PRLR dimerization and the consequent recruitment of one or more associated JAK tyrosine kinases (mainly JAK2), which causes trans-phosphorylation of JAK kinases and subsequent phosphorylation of PRLR. Phosphorylated JAKs subsequently phosphorylate STAT transcription factors (mainly STAT5) which dimerize and become able to translocate to the nucleus where they activate target genes (Bole-Feysot et al (1998) Endocr. Rev. 19:225-268).

In humans, it has been shown that PRL is also synthesized in many extrapituitary sites, such as mammary epithelial cells. PRL exerts a proliferative action on these cells, which express PRLR, via an antocrine/paracrine loop. It has thus been suggested that the growth-promoting activity exerted by PRL on some target tissues under normal conditions may be somehow involved in promoting tumor growth under pathological conditions. Nevertheless, implication of the PRL axis in breast diseases is a matter of debate. The role of PRL in breast tumorigenesis remains difficult to affirm, despite wide epidemiological studies showing that high-normal PLR levels are associated with an increased risk of breast cancer (Tworoger and Hankinson (2008). J. Mammary. Gland. Biol. Neoplasia. 13:41-53). Unfortunately, data from large cohorts of hyperprolactinemic patients are lacking to further support this association. In contrast, in rodent models systems, it has been shown that PRL plays a key role in the development of mammary cancer (Goffin et al (2005) Endocr. Rev. 26:400-422).

A human genetic model has yet to be identified to confirm the role of PRUPRLR in breast diseases. Four studies focused on the identification of PRL and/or PRLR gene mutations in breast cancer patients, and their potential association with the risk to develop the disease (Glasow et al. (2001) J. Clin. Endocrinol. Metab. 86:3826-3832; Canbay et al. (2004) Curr. Med. Res. Opin. 20:533-540; Vaclavicek et al. (2006) J. Clin. Endocrinol. Metab. 91:1513-1519; Lee et al. (2007) BMC Med. Gen. 8:72). While several non coding single nucleotide polymorphisms (SNPs) and fewer silent SNPs (located in exons) were reported in both genes, not necessarily in patients only, no more than two missense SNPs could be identified so far in the PRLR gene, which both involve residues of the extracellular domain: substitution of Valine for Isoleucine 76 (I76V, reported first in NCBI database, and later in an epidemiological study where this SNP was referred to as Ile100Val according to sequence numbering including signal peptide; Lee et al. (2007) BMC Med. Gen. 8:72) and of Leucine for Isoleucine 146 (I146L, first reported in Canbay et al. (2004) Curr. Med. Res. Opin. 20:533-540). No functional investigation was conducted in these studies to determine whether the amino acid substitutions eventually modified the properties of the encoded receptor variant. Additionally, no breast cancer risk could be definitely associated with these individual SNPs. Indeed, although Canbay et al. suggested a potential role for PRLR-I146L based on the fact that it was discovered in two patients among the 38 patients analyzed and in none of the 100 control subjects, no statistical evidence was provided. Furthermore, no association with a particular type of breast cancer was demonstrated nor suggested.

In parallel, the present inventors identified three germline, heterozygous missense SNPs in the PRLR gene of patients suffering from multiple breast fibroadenoma (MFA), a rare benign disease that is defined by the presence of at least three lesions (adenomas) simultaneously in one breast. Among these mutations, two mutations, the mutation I146L and the mutation I76V, occurred at a higher frequency in MFA patients than in control subjects. In particular, the mutation I146L was observed in MFA patients (4 cases among 95) but not in control subjects (see international patent application WO2008/114077, Bogorad et al. (2008) Proc. Natl. Acad. Sci. 105:14533-14538 and Courtillot et al. (2010) J. Clin. Endocrinol. Metab. 95:271-279). The present inventors demonstrated that these sole substitutions were sufficient to confer constitutive activity to the receptor variant (Bogorad et al. (2008) Proc. Natl. Acad. Sci. 105:14533-14538; Courtillot et al. (2010) J. Clin. Endocrinol. Metab. 95:271-279). Nevertheless, no potential link between fibroadenoma and subsequent breast cancer has been clearly demonstrated by now (Worsham et al. (2009) Breast Cancer Res. Treat. 118:1-7). In particular, no link between fibroadenoma and subsequent triple-negative has been searched until now.

The present invention arises from the unexpected finding by the inventors that, when stably expressed in breast cancer cells, constitutively activated PRLR-I146L increases their aggressive potential, which can be observed by (i) an increased autonomous proliferation, (ii) the acquisition of migratory and invasive properties, (iii) a hormone-independence associated with the loss of expression of the estrogen receptor and progesterone receptor (i.e. a hormone-receptor negative breast cancer), associated either with a normal or abnormal low expression (also called “under-expression”) of HER2, or with the absence of detectable HER2 receptor (in particular by immunohistochemistry) (defining the triple-negative cancers ER−/PR−/HER2−), or associated with an over-expression of HER2 (defining the hormone receptor negative (i.e. absence of estrogen and progesterone receptors (ER−/PR− profile), HER2₊ breast cancers), (iv) the loss of epithelial characteristics in favor of mesenchymal characteristics and (v) the acquisition of characteristics of cancer stem cells. The inventors further confirmed the aggressiveness of PRLR-I146L-expressing cells by xenograft experiments in immunodepressed mice, observing their metastatic potential which is absent from parental cells.

The present inventors therefore demonstrated that the presence of a mutant PRLR displaying a constitutive activity, such as PRLR-I146L or PRLR-I76V, was a predictive marker of tumoral progression of breast cancer, in particular of a progression towards a hormone-receptor negative (including triple-negative) breast cancer.

The present invention thus relates to an in vitro method of prognosing outcome of hormone-dependent cancer, such as breast cancer or prostate cancer, in a subject comprising detecting a constitutively activating mutation in the prolactin receptor (PRLR) gene in a nucleic acid sample previously obtained from said subject.

### Hormone-Dependent Cancers

As used herein, the term “hormone-dependent cancer” refers to a cancer that has a hormonal sensitivity. Hormone-dependent cancers are well-known from the skilled person and include in particular breast cancer and prostate cancer. When the term “hormone-dependent cancer” is used in association with breast and prostate cancers, it means that the cancers have sensitivity to steroid hormones, i.e. estrogen and progesterone for breast cancers and androgen for prostate cancers. Hormone sensitive cancers respond to anti-hormone therapies targeting these steroid hormone receptors.

As used herein, the term “breast cancer” relates to any of various carcinomas of the breast or mammary tissue.

Breast cancers can be distinguished according to their stage, their pathology, their grade, their receptor status and their gene expression pattern.

Depending on their stage, i.e. on the size of the tumor, on whether or not the tumor has spread to the lymph nodes in the armpits, and on whether or not the tumor has metastasized, breast cancers may be divided into breast cancer of stage 0, which is a pre-malignant disease, breast cancer of stage 1-3 defined as “early” cancer, and breast cancer of stage 4, defined as “advanced” and/or “metastatic” cancer.

Depending on their pathology, i.e. on the tissues from which breast cancer derives, breast cancers may be divided into carcinoma in situ, defined as a proliferation of cancer cells within the epithelial tissue without invasion of the surrounding tissue, and invasive carcinoma, which invades the surrounding tissue.

Depending on their grade, i.e. on the differentiation stage of cancer cells, breast cancers may be divided into low grade breast cancer, wherein cells are well differentiated, intermediate grade breast cancer, wherein, cells are moderately differentiated, and high grade breast cancer, wherein cells are poorly differentiated.

Depending on their receptor status as detected by immunohistochemistry (see for instance the method disclosed by M. Elizabeth H et al. (Journal of Clinical Oncology, 28(16): 2784-2795, 2010) for determining status of estrogen and progesterone receptors, and the semi-quantitative immunohistochemical assay “HercepTest™” manufactured by Dako for determining overexpression of HER2), in particular on the presence or absence of estrogen receptor (ER), progesterone receptor (PR) and on the level of expression of HER2/neu (normal expression/under-expression vs over-expression), breast cancers may be divided into ER positive (ER+) breast cancer, ER negative (ER−) breast cancer, PR positive (PR+) breast cancer, PR negative (PR−) breast cancer, HER2 positive (HER2+) breast cancer (cancer over-expressing HER2), HER2 negative (HER2−) breast cancer (cancer expressing normal levels of HER2 or under-expressing HER2, or not expressing a detectable level of HER2), hormone receptor negative breast cancer, i.e. breast cancer with neither of estrogen nor progesterone receptors (abbreviated by ER−/PR− breast cancer); and triple negative breast cancer, i.e. breast cancer with neither of estrogen nor progesterone receptors and with normal expression/under-expression (or with the absence of detectable level of expression) of HER2 (abbreviated by ER−/PR−/HER2− breast cancer).

Depending on their gene expression pattern, breast cancers may be divided into luminal subtype A breast cancer, luminal subtype B breast cancer, normal-like breast cancer, HER2+ breast cancer and basal-like breast cancer (Sorlie et al. (2001) Proc. Nat. Acad. Sci. 98:10869-10874). Luminal A and B subtypes are largely ER positive. In contrast, HER2+ breast cancers show an increased high expression of genes associated with the HER2 amplicon and normal-like breast cancers share molecular features of normal breast tissue.

Basal-like breast cancers (BLBC) are characterized by the lack of expression of ER, PR, and the absence of detectable level of expression of HER2 or the normal expression or under-expression of HER2 (i.e. triple negativity) as well as an increased expression of basal (myoepithelial) cytokeratins such as CK14, CK5/6 and CK17, of p63 and epidermal growth factor (EGFR). BLBC arise from the outer (basal) layer of normal breast ducts (i.e. myoepithelial cells) or originate from a stem cell precursor of basal myoepithelial cells. Furthermore, BLBC exhibit well-established characteristics of epithelial-mesenchymal transition, such as loss of epithelial characteristics and acquisition of a mesenchymal phenotype (Sarrió et al. (2008) Cancer Res. 68:989-997).

As used herein, the expression “epithelial-mesenchymal transition” or “EMT” refers to a developmental process by which cells of epithelial origin lose epithelial characteristics and polarity, and acquire a mesenchymal phenotype with increased migratory behavior. EMT is in particular characterized by loss of intercellular adhesion (in particular E-cadherin and occludins), down-regulation of epithelial markers such as cytokeratins, up-regulation of mesenchymal markers such as vimentin and smooth muscle actin, acquisition of fibroblast-like or spindle like morphology with cytoskeleton reorganization, and increase in motility, invasiveness and metastatic capabilities.

As used herein, the term “triple-negative breast cancer” refers to a breast cancer which is characterized by a lack of detectable expression of both ER and PR (preferably when the measures of expression of ER and PR are carried out by the method disclosed by M. Elizabeth H et al., Journal of Clinical Oncology, 28(16): 2784-2795, 2010), as well as by a standard (normal) expression, an under-expression of HER2 or the absence of detectable level of expression of HER2.

As used herein, the terms “normal expression of HER2” and “standard expression of HER2” can be used indifferently, and means the level of expression of HER2 of a healthy cell which does not comprise amplification of HER2 gene. Using the semi-quantitative immunohistochemical assay “HercepTest™” manufactured by Dako for determining of HER2, a level of expression scoring 0 or 1+, or 2+ with negative fluorescence in situ hybridization, is representative of a normal expression of HER2.

Triple-negative breast cancer may also be characterized by a distinct molecular profile when compared with hormone receptor positive breast cancers. In particular, triple-negative breast cancer may be characterized by a differential expression of 269 of 371 genes associated with kinase activity, cell division, proliferation, intracellular DNA repair, antiapoptosis and transcriptional regulation described by Sparano et al. (Sparano et al. (2009) J. Clin Oncol. (Meeting Abstracts) 27: (Abstr 500)).

As used herein, the term “hormone-dependent stage” of a breast cancer refers to a breast cancer wherein the cancer cells express a hormone receptor, in particular estrogen receptor or progesterone receptor, and are responsive to these hormones. Hormone-dependent stage of breast cancers may generally be treated by anti-hormonal strategies.

As used herein, the term “hormone-independent stage” of a breast cancer refers to a breast cancer wherein the cancer cells do not detectably express hormone receptors, such as estrogen receptors or progesterone receptors. Hormone-independent stages of breast cancers are generally not treatable by anti-hormonal strategies.

As used herein, the term “metastatic cancer” refers to a cancer which spreads or will spread beyond the original organ. Metastatic cancer will cause symptoms that depend on the location of metastasis. Common sites of metastasis of metastatic breast cancer include bone, liver, lung and brain.

As used herein, the term “prostate cancer” refers to any of various carcinomas of the prostate.

Prostate cancers can be distinguished in particular according to their stage and their grade.

Depending on their stage, i.e. on the size of the tumor, on whether or not the tumor has spread to the lymph nodes, and on whether or not the tumor has metastasized, prostate cancers may be divided into prostate cancer of stage 0, which is a pre-malignant disease, prostate cancer of stage 1-3 defined as “early” cancer, and prostate cancer of stage 4, defined as “advanced” and/or “metastatic” cancer.

Depending on their grade, i.e. on the difference of cancer cells from normal prostate cells, prostate cancers may be classified from grade 2 to grade 10 according the Gleason system, where a Gleason score of 10 indicates the most abnormalities.

Prostate cancers can also be distinguished according to their hormonal sensitivity. Hormone-dependent prostate cancers have sensitivity to androgen, whereas hormone-independent prostate cancers have no sensitivity to androgen, for instance following the loss of expression of androgen receptors or the occurrence of mutation in the androgen receptor gene.

### Subject

In the context of the present invention, a “subject” denotes a human or non-human mammal, such as a rodent (rat, mouse, rabbit), a primate (chimpanzee), a feline (cat), a canine (dog). Preferably, the subject is human, in particular a male or a female. Still preferably, the subject is a female.

In a particular aspect of the present invention, the subject may be a subject suffering from hormone-dependent cancer, in particular from breast cancer or prostate cancer, preferably from breast cancer. Preferably, the subject is a subject who has been diagnosed as suffering from hormone-dependent cancer, in particular from breast cancer or prostate cancer, preferably from breast cancer.

In a particular embodiment, the subject is a subject suffering or who suffered from a hormone-dependent stage of breast cancer.

In another particular embodiment, the subject is a subject suffering from a hormone-independent stage of breast cancer or from a metastatic breast cancer, in particular from a basal-like breast cancer or a triple-negative breast cancer.

### Mutations in the PRLR Gene

In the context of the invention, the term “prolactin receptor” or “PRLR” refers to a transmembrane protein that belongs to the cytokine receptor superfamily and is expressed in a wide variety of tissues in addition to the breast. It is closely related to growth-hormone receptor. Human PRLR is encoded by a single gene located on chromosome 5 and which is approximately 180 kb in length. It was originally described as having 10 exons, before exon 11 was identified (Hu et al. (2001) J. Biol. Chem. 276:41086-41094). Exons 3-11 are coding exons. The sequence of the cDNA encoding human PRLR is in particular shown in SEQ ID NO: 1. PRLR consists of three parts or domains: an extracellular domain of about 210 amino acids, a transmembrane domain of about 24 amino acids, and an intracellular domain. The amino acid residues which delineate these domains are for example described in Kelly et al. (1989) Biol Reprod. 40:27-32. The extracellular ligand-binding domain folds into two β-sheets each containing seven β-strands. Although the PRLR gene is unique in each species, various isoforms resulting from alternative splicing have been described. They only differ in the length and composition of their intracellular tail, while their extracellular ligand-binding domain is identical. The amino acid sequence of human PRLR precursor is typically shown in SEQ ID NO: 2. The active form of PRLR is a homodimer composed of two identical membrane chains, each of which interacts with opposing sides of prolactin, referred to as binding sites 1 and 2. PRLR transduces the signal via associated kinases, which are recruited by the receptor, when not pre-bound to the receptor, and activated upon ligand binding. Several tyrosine or serine/threonine kinases are involved in PRLR signaling, among which JAK, MAPK and Src which each trigger specific cascades.

The present inventors have demonstrated that a particular mutation in the PRLR gene inducing a constitutive activity of PRLR was predictive of the progression of a hormone-dependent cancer towards an aggressive, metastatic, hormone-independent cancer. In particular, in the case of breast cancer, they have demonstrated that this particular mutation was predictive of the progression of breast cancer towards an aggressive, triple-negative breast cancer.

They have identified two particular mutations in the PRLR gene, responsible for such a constitutive activity of PRLR. Such a mutation is called herein a constitutively activating mutation. One of these mutations is localized in exon 6 of the PRLR gene. It is a nonsynonymous A to C substitution at position 821 of the PRLR mRNA (GenBank NM 000949), resulting in a mutant prolactin receptor having an isoleucine (Ile, ATT) to leucine (Leu, CTT) substitution at position 146 in the polypeptide sequence of the mature form of the receptor (position 170 in the polypeptide sequence of the unprocessed precursor which is available for example as Swiss-Prot entry P16471-1). This mutation will be designed hereinafter as I146L. The other mutation is localized in exon 5 of the PRLR gene. It is a nonsynonymous A to G substitution at position 611 of the PRLR mRNA, resulting in a mutant prolactin receptor having an isoleucine (Ile, ATC) to valine (Val, GTC) substitution at position 76 in the polypeptide sequence of the mature form of the receptor (position 100 in the polypeptide sequence of the unprocessed precursor).

The positions indicated herein refer to the sequence of the mature form of the human prolactin receptor, which is represented in SEQ ID NO: 3.

As shown in Bogorad et al. (2008) Proc. Natl. Acad. Sci. 105:14533-14538 and in Courtillot et al. (2010) J. Clin. Endocrinol. Metab. 95:271-279, these amino acid substitutions generate constitutively active PRLR variants.

As used herein, the expressions “constitutively active PRLR variant”, “constitutively active mutant of the prolactin receptor” or “mutant PRLR displaying a constitutive activity” are used interchangeably and refer to a mutant of said receptor exhibiting a biological activity (i.e. triggering downstream signaling) in the absence of prolactin stimulation, and/or which is higher than the biological activity of the corresponding wild-type receptor in the presence of prolactin. A constitutively active PRLR variant according to the invention is in particular a mutant wherein the isoleucine residue at position 146 is substituted with leucine (named herein below I146L) or a mutant wherein the isoleucine residue at position 76 is substituted with valine (named herein below I76V). Preferably, a constitutively active PRLR variant according to the invention is a mutant wherein the isoleucine at position 146 is substituted with leucine.

In the context of the invention, the expression “constitutively activating mutation” refers to a mutation resulting in the expression of a constitutively activated variant as defined above.

Methods for determining whether a mutation of the PRLR gene is a constitutively activating mutation according to the invention are for example described in Bogorad et al. (2008) Proc. Natl. Acad. Sci. 105:14533-14538 and in Courtillot et al. (2010) J. Clin. Endocrinol. Metab. 95:271-279. Typically, cells, such as Ba/F3 pro-B lymphoid cells, are transfected using expression vector for mutant PRLR. Stable clones or populations are then selected by addition of an antibiotic in a medium containing growth factors that are necessary for the survival and/or proliferation of the cells. Survival/proliferation assays are then performed by incubating each stable population with or without the above cited growth factors and prolactin. The survival and/or the proliferation of a stable population in the absence of said growth factors and prolactin indicates that the expressed mutant PRLR constitutively activates downstream signaling cascades required for survival/proliferation, meaning that the mutation is a constitutively activating mutation.

The constitutively activating mutation may be a germline mutation, i.e. a mutation present in the genome of the subject at the heterozygous or homozygous state. The constitutively activating mutation may also be a somatic mutation, i.e. a mutation only found in the tumor of the subject.

In Vitro Method of Prognosing Outcome of Hormone-Dependent Cancer

The present invention therefore relates to an in vitro method of prognosing outcome of hormone-dependent cancer, in particular of breast cancer or prostate cancer, in a subject comprising detecting a constitutively activating mutation in the prolactin receptor (PRLR) gene in a nucleic acid sample previously obtained from said subject.

Preferably, said constitutively activating mutation is a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine. More preferably, said constitutively activating mutation is a mutation resulting in the expression of a mutant prolactin receptor wherein the isoleucine residue at position 146 is substituted with leucine.

As used herein, the term “prognosing” refers to the determination or prediction of the probable course of a disease.

As used herein, the term “outcome” or “clinical outcome” refers to the health status of a subject following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, progression of the disease, metastasis, advanced or aggressive disease, disease recurrence and death. Preferably, the outcome of hormone-dependent cancer according to the invention is the progression of hormone-dependent cancer in the subject.

As used herein, the term “progression of cancer” or “cancer progression” refers to any measure of cancer growth, development, and/or maturation including metastasis. “Cancer progression” includes increase in cell number, cell size, tumor size, and number of tumors, as well as morphological and other cellular and molecular changes and other staging characteristics. More particularly, progression of a hormone-dependent cancer, in particular of breast cancer or prostate cancer, includes the evolution of a cancer from a hormone-dependent stage towards a hormone-independent stage, and the evolution of a cancer towards a metastatic cancer. In particular, when the hormone-dependent cancer is breast cancer, such a progression includes the evolution of a breast cancer towards a hormone receptor negative cancer, a basal-like or triple-negative cancer and the evolution of a breast cancer towards a metastatic breast cancer.

Accordingly, in a particular embodiment, the method as defined above is for prognosing progression of the hormone-dependent cancer, in particular of the breast cancer or prostate cancer, from a hormone-dependent stage towards a hormone-independent stage. In another particular embodiment, the method as defined above is for prognosing progression of the breast cancer towards a hormone receptor negative cancer. In another particular embodiment, the method as defined above is for prognosing progression of the breast cancer towards a basal-like or a triple-negative breast cancer. As explained above in the section “Hormone-dependent cancer”, basal-like breast cancers exhibit well-established characteristics of epithelial-mesenchymal transition. Therefore, in another particular embodiment, the method as defined above is for prognosing an epithelial to mesenchymal progression of breast cancer cells. Furthermore, the above types of breast cancer display metastatic properties. Accordingly, in another particular embodiment, the method as defined above is for prognosing progression of the cancer towards a metastatic cancer.

The presence of a mutation in a gene can be detected by any of a number of diagnostic assays. These assays may use otherwise known techniques, including direct sequencing of the nucleic acids in the sample, or using probes which overlap the position of the SNP on those nucleic acids. Techniques for detecting the mutation according to the invention also include array-based sandwich assays, ligase-based methods, mass-spectroscopy-based methods, PCR-based methods, exonuclease-based methods, dideoxynucleotide-based methods, Genetic Bit Analysis or GBA, Oligonucleotide Ligation Assays or OLAs, and primer-guided nucleotide incorporation procedures. SNP detection by means of an array-based sandwich assay is for example described in U.S. Pat. No. 6,410,231. This document also makes mention of a variety of other techniques that had been previously developed for SNP detection and analysis such as those described in U.S. Pat. No. 5,858,659; U.S. Pat. No. 5,633,134; U.S. Pat. No. 5,719,028; international application WO 98/30717; international application WO 97/10366; international application WO 98/44157; international application WO 98/20165; international application WO 95/12607 and international application WO 98/30883. In addition, suitable ligase based methods are described in the international application WO 97/31256 and Chen et al. (1998) Genome Res. 8:549-556. Suitable mass-spectroscopy-based methods are disclosed in international application WO 98/12355 and international application WO 98/14616. Suitable PCR-based methods are disclosed by Hauser et al. (1998) Plant J. 16:117-125. Suitable exonuclease-based methods are described in U.S. Pat. No. 4,656,127. Suitable dideoxynucleotide-based methods are described in the international application WO 91/02087. Genetic Bit Analysis or GBA is described in the international application WO 92/15712. Oligonucleotide Ligation Assays or OLAs are described in Landegren et al. (1988) Science 241:1077-1080 and Nickerson et al. (1990) Proc. Natl. Acad, Sci. (USA) 87:8923-8927. Primer-guided nucleotide incorporation procedures are described by Prezant et al. (1992) Hum. Mutat. 1:159-164; Ugozzoli et al. (1992) GATA 9:107-112 and Nyreen et al. (1993) Anal. Biochem. 208:171-175. Other potential assay techniques are described in U.S. Pat. No. 6,340,566, which teaches detection and quantification of SNP, DNA sequence variations, DNA mutations, DNA damage and DNA mismatches using mutation binding proteins alone or as chimeric proteins with nucleases on solid supports; in U.S. Pat. No. 6,376,177, which teaches a method and apparatus for SNP detection by means of spectroscopic analysis of hybridized nucleic acid using high density nucleic acid chips. Numerous conventional assay techniques for detecting SNP which are also suitable for use herein are described in U.S. Pat. No. 6,428,958 and U.S. Pat. No. 6,355,425.

In accordance with one preferred embodiment, the presence of the I146L mutation may be detected as described in Bogorad et al. (2008) Proc. Nat. Acad. Sci. 105:14533-14538 by entirely sequencing the exon 6 of the PRLR gene in both directions using primers matching intronic boundaries, or by performing an ApoI enzymatic digestion on the PCR amplification fragment wherein the mutation is looked for. Similarly, the presence of the I76V mutation may be detected by entirely sequencing the exon 5 of the PRLR gene in both directions using primers matching intronic boundaries, or by restriction with the restriction enzyme HpyCH4 IV.

Nucleic acid samples suitable for performing the detection include mRNA, cDNA or genomic DNA. Since the constitutively activating mutation which is looked for may be a germline mutation, nucleic acid samples previously obtained from the subject need not to directly derive from tumor cells. Accordingly, nucleic acid samples obtained from the subject may be tumoral nucleic acid samples or non-tumoral nucleic acid samples. Preferably, the nucleic acid sample is obtained from whole blood cells from the subject.

The expression of a constitutively active mutant of the PRLR may further be confirmed by quantifying the activated form of PRLR in a biological sample previously obtained from the subject. This quantification can be for example performed by measuring the quantity of phosphorylated PRLR with an antibody able to differentiate between the phosphorylated and non-phosphorylated forms of PRLR. This can be performed by immunohistochemical analyses of tumor samples obtained from the patients, or by analysis (western blot or other method) of PRLR activation in any cells/tissues (harvested from the patients) which are known to express the PRLR, e.g. lymphocytes.

The present invention also relates to an in vitro method of prognosing outcome of hormone-dependent cancer, in particular of breast cancer or prostate cancer, in a subject comprising

(i) detecting a constitutively activating mutation in the prolactin receptor (PRLR) gene in a nucleic acid sample previously obtained from said subject, said mutation preferably being a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine; and

(ii) deducing therefrom the outcome of hormone-dependent cancer in the subject.

In particular, when the in vitro method is carried out to prognose outcome of a hormone-dependent breast cancer, the presence of said constitutively activating mutation is indicative of a progression of breast cancer towards a hormone-independent stage, in particular a hormone-receptor negative cancer, a basal-like or triple-negative breast cancer.

When the in vitro method is carried out to prognose outcome of a hormone-dependent prostate cancer, the presence of said constitutively activating mutation is indicative of a progression of prostate cancer towards hormone-independent prostate cancer (i.e. prostate cancers which have no sensitivity to androgen, for instance following to loss of expression of androgen receptors).

In the context of the invention, the subject, for which outcome of hormone-dependent cancer is prognosed, may be heterozygous or homozygous for the mutation, i.e. both alleles of the PRLR gene in the subject may contain the constitutively activating mutation, or only one allele the PRLR gene in the subject may contain the constitutively activating mutation.

Inhibitor of PRLR-triggered signaling cascades and uses thereof. Beyond the prognostic feature of the detection of the constitutively activating mutations identified by the inventors, this analysis has an impact on the therapeutic strategy proposed to the patients by blocking the constitutive signaling of the mutated receptor or by blocking pathways activated downstream thereof.

Accordingly, the present invention also relates to an inhibitor of PRLR-triggered signaling cascades for use for treating hormone-dependent cancer, in particular breast cancer or prostate cancer, and/or preventing progression of hormone-dependent cancer, in particular breast cancer or prostate cancer, towards an hormone-independent stage in a subject. In a preferred embodiment, the presence of a constitutively activating mutation in the PRLR gene has been detected in a nucleic acid sample previously obtained from said subject. Preferably, said constitutively activating mutation is a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine. More preferably, said constitutively activating mutation is a mutation resulting in the expression of a mutant prolactin receptor wherein the isoleucine residue at position 146 is substituted with leucine.

In the context of the invention, the expression “PRLR-triggered signaling cascades” refers to the cascade of intracellular events activated by the interaction of prolactin with the prolactin receptor. PRLR-triggered signaling cascades are for example described in Bole-Feysot et al. (1998) Endocrine Rev. 19:225-268. In particular, PRLR-triggered signaling cascades include activation of at least one of the genes selected from the group consisting of IRF-1, c-myc, c-fos, ornithine decarboxylase (ODC), hsp 70, β-Actin, pim-1, gfi-1, bcl-2, bax, T-cell receptor γ-chain, cyclins D2 and D3, cyclin E, cdk2, cdk5, E2F-1, clone 15 nuclear movement protein, GnRH, GnRH receptor, vimentin, laminin, caveolin 1, caveolin 2, Snail1 and Snail2. PRLR-triggered signaling cascades also include inactivation of at least one of the genes selected from the group consisting of N-cadherin, E-cadherin, P-cadherin, keratin 18, keratin 19 and fascine. PRLR-triggered signaling cascades also include activation of at least one of the proteins selected from the group consisting of JAK2, Stat proteins, Src, Fyn, Ras, Raf, Vav, Grb2, Sos, Shc, MAP kinase (for instance p38, MEK and MEK2), Stathmin, IRS-1, PTP-1D (SHP-2), PKC, casein kinase II, PTK, PI3 kinase, Akt, mTOR, cbl, S6 kinase, G proteins, PLC and amiloride-sensitive Na⁺/H⁺ exchange system. Preferably, the PRLR-triggered signaling cascades according to the invention encompass the activation of the JAK/Stat pathway, of the Ras/Raf/MAP kinase pathway and of the Src kinase pathway. More preferably, the PRLR-triggered signaling cascade according to the invention is the activation of the JAK/Stat pathway.

In the context of the invention, the expression “inhibitor of PRLR-triggered signaling cascades” refers to a compound that prevents and/or decreases the activation of the cascade of intracellular events activated by the activation of prolactin receptor. In particular, inhibitors of PRLR-triggered signaling cascades may be compounds that prevent and/or decrease the activation of the cascade of intracellular events activated by the interaction of prolactin with the prolactin receptor and/or compounds that prevent and/or decrease the activation of the cascade of intracellular events activated by a constitutively activated prolactin receptor variant. Preferably, inhibitors of PRLR-triggered signaling cascades according to the invention are compounds that prevent and/or decrease the activation of the cascade of intracellular events activated by a constitutively activated prolactin receptor variant.

Such an inhibitor of PRLR-triggered signaling cascades may be chosen among the JAK2 inhibitors. Preferably, the JAK2 inhibitor is selected from the group consisting of the compounds 1 to 98 disclosed in FIGS. 1 to 18 of the review article by Robert Kiss et al. (Expert Opin. Ther. Patents, 20(4): 471-495, 2010), compound called “AZD1480” developed by AstraZeneca and compound called “WP1066” (also known as (E)-3(6-bromopyridin-2-yl)-2-cyano-N-(S0-1-phenylethyl)acrylamide). More preferably the JAK2 inhibitor is the compound called “AG490” or one of its analogues (compounds 1 to 12 disclosed in FIG. 1 of the review article by Robert Kiss et al. (2010).

Such an inhibitor of PRLR-triggered signaling cascades may also be chosen among the MAP kinase inhibitors, for instance the p38 MAP kinase inhibitors and the inhibitors of MAP kinases MEK and MEK2. Such inhibitors may be in particular the inhibitors disclosed in the review article by Hae-Young Yong et al. (Expert Opin. Investig. Drugs, 18(12): 1893-1905, 2009) and in the review article by P. J. Roberts and C. J. Der (Oncogene, 26: 3291-3310, 2007). Preferably, the p38 MAP kinase inhibitor is selected from the group consisting of compounds SB203580, SB239063, SB20025, SB202190, SB242235, RWJ67657, SD282, BIRB-796, R-130823, Talmapimod (SC10-469) (see Table 2 of Hae-Young Yong et al. (2009), RO4402257, RO3201195, PH-797804, AZD-6703, SB-681323, VX-745, VX-702, Scio-469, Scio-323, TAK-715, PS540446, RWJ-67657, BIRB-796, KC706, ARRY-797 and AMG-548 (see Table 2 of P. J. Roberts and C. J. Der (2007). Preferably, the inhibitor of MEK and MEK2 is selected from the group consisting of compounds PD0325901, AZD6244 and ARRY-438162 (see Table 2 of P. J. Roberts and C. J. Der (2007).

Such an inhibitor of PRLR-triggered signaling cascades may also be chosen among the mTOR inhibitors, in particular the inhibitors disclosed in the review article by Fasolo and Sessa (Expert Opin. Investig. Drugs, 17(11): 1717-1734, 2008). Preferably, the mTOR inhibitor is selected from the group consisting of the compounds CCI-779 (Temsirolimus), RAD001 (Everolimus) and AP23573 (Defololimus) (see Table 1 of the review article by Fasolo and Sessa (2008)).

Inhibitors of PRLR-triggered signaling cascades according to the invention may be in particular PRLR antagonists, PRLR inverse agonists and/or inhibitors of downstream activated receptor-associated kinases.

In a particular embodiment, the inhibitor of PRLR-triggered signaling cascades is a PRLR antagonist.

As used herein, the expression “PRLR antagonist” refers to a compound that binds but does not activate PRLR, thereby preventing prolactin from activating the PRLR-triggered signaling cascades defined above, preferably by a competitive mechanism. In particular, PRLR antagonists include inhibitory prolactin variants, as defined herein below, and neutralizing antibodies specifically directed against PRLR.

Examples of PRLR antagonists are well-known from the skilled person and are for example described in Goffin et al. (2005) Endocr. Rev. 26:400-422 and Goffin et al. (2007) Recent Patents on Endocrine, Metabolic & Immune Drug Discovery 1:41-52. Examples of PRLR antagonists that can be used according to the invention include those disclosed in the international application WO 03/057729, which are variants of prolactin having mutations preventing the formation of the disulfide bridge between Cys₄ and Cys₁₁, and inducing steric hindrance within binding site 2 of prolactin; those disclosed in the international application WO 2009/022235, which are variants of prolactin having a sterically hindering mutation within binding site 2 of prolactin; and those described in Chen et al. (1998) Endocrinology 139:609-616, which are variants of prolactin which mimic the phosphorylated form of prolactin.

Accordingly, in a preferred embodiment, the PRLR antagonist is selected from the group consisting of

(a) a variant of prolactin comprising a mutation consisting of the substitution of the glycine residue at position 129 with any amino acid residue other than glycine, with the exception of proline, and

(b) a variant of prolactin comprising:
(i) a mutation or a set of mutations within the 14 N-terminal amino acid residues, wherein said mutation or set of mutations prevents the formation of the disulfide bridge between the cystein at position 4 and the cystein at position 11, and
(ii) a sterically hindering mutation or set of mutations within binding site 2 of prolactin.

Positions indicated herein above refer to the amino acid numbering of prolactin under its mature form, devoid of the signal peptide. Advantageously the variants of prolactin defined above are variants of human prolactin (hPRL). The sequence of unprocessed precursor of hPRL is available for example in the Swiss-Prot database under accession number P01236. It is typically shown in SEQ ID NO: 4.

In the variants of prolactin defined herein above in (a), the amino acid substituting Gly₁₂₉ is selected from the group consisting of alanine, asparagine, aspartic acid, arginine, cysteine, glutamic acid, glutamine, histidine, isoleucine, leucine, lysine, methionine, phenylalanine, serine, threonine, tryptophan, tyrosine and valine, preferably asparagine, arginine, leucine, phenylalanine and valine, more preferably arginine, phenylalanine, leucine and valine.

In the variants of prolactin defined herein above in (b), mutation(s) (i) impairing the formation of the Cys₄-Cys₁₁ disulfide bridge comprise for example deletions including Cys₄ and/or Cys₁₁, or substitution of Cys₄ and/or Cys₁₁ by an amino acid other than cystein. Mutation(s) (ii) comprise in particular any substitution of a small amino acid within binding site 2 of PRL by a large and/or charged amino acid in order to introduce a steric hindrance. Examples of such mutations are for instance substitution of at least one residue among Gln₂₂, Leu₁₂₅, Ser₂₆, Ala₂₂ or Gly₁₂₉ by residues such as tyrosine, phenylalanine, aspartic acid, glutamic acid, arginine, lysine or tryptophan.

More preferably, the PRLR antagonist according to the invention is selected from the group consisting of

(a) a variant of prolactin comprising a mutation consisting of the substitution of the glycine residue at position 129 with an arginine residue (G129R-PRL), a phenylalanine residue (G129F-hPRL), a leucine residue (G129L-hPRL) or a valine residue (G129V-PRL),

(b) a variant of prolactin comprising a deletion of at least the 9 N-terminal amino acid residues and up to the 14 N-terminal amino acid residues and a substitution of the glycine residue at position 129 with an arginine residue.

Methods for identifying inhibitors of PRLR antagonists are well-known from the skilled person and include assays to test the ability of a compound to antagonize PRL activity in a panel of cell lines expressing different levels of PRLR and/or PRL, so as to permit the inference of an effect which varies according to PRL/PRLR availability. For example, the activity of a PRLR antagonist may be tested in all or a subset of the following five different human breast cancer cell lines (T-47D, MCF-7, HTB 19, HTB20, and HTB 123 from ATCC). The PRLR numbers on these cell lines have been reported to be: T-47D (25,800/cell), MCF-7 (8,300/cell), HTB19 (6,435/cell), HTB20 (5,480/cell), HTB123 (1,094/cell, normal breast cell=1,700/cell). Therefore, these cell lines represent a spectrum of PRLR levels on human breast cancer cells. It should be noted that the use of human breast cancer cell lines is preferred over the use of the rat Nb2 T-cell lymphoma cell line, widely used in the lactogenic hormone studies, in order to avoid the potential confusing effects caused by species specificity. Assays which may be used to determine the effects of the PRLR antagonists include (i) a competitive receptor binding assay, (ii) detection/quantitation of phosphorylation of STAT5 protein and (iii) a cell proliferation assay.

In another particular embodiment, the inhibitor of PRLR-triggered signaling cascades is a PRLR inverse agonist.

As used herein, the expression “PRLR inverse agonist” refers to a compound that binds to the same receptor binding-site as an agonist for PRLR and reverses constitutive activity of mutated PRLR. Examples of PRLR inverse agonists are described in Bogorad et al. (2008) Proc. Nat. Acad. Sci. 105:14533-14538. They include in particular 41-9-G129R-hPRL.

Methods for identifying PRLR inverse agonists are well-known from the skilled person and are for example described in Bogorad et al. (2008) Proc. Nat. Acad. Sci. 105:14533-14538. Typically, such method comprises (i) incubating the candidate compound with cells stably expressing a constitutively activated mutant of PRLR, such as PRLR I146L; (ii) determining the phosphorylation state of the constitutively activated mutant of PRLR and/or of signalling target molecules such as Stat5; wherein, if the candidate compound inhibits phosphorylation of the constitutively activated mutant of PRLR and/or of signalling target molecules such as Stat5, said candidate compound is a PRLR inverse agonist.

As known from the skilled person, an inhibitor of PRLR-triggered signalling cascades may be both a PRLR antagonist and a PRLR inverse agonist. Such an inhibitor of PRLR-triggered signalling cascades is for example Δ1-9-G129R-hPRL.

In still another particular embodiment, the inhibitor of PRLR-triggered signaling cascades is an inhibitor of a downstream activated receptor-associated kinase.

As highlighted above, kinases downstream activated by PRLR are well-known from the skilled person. “Downstream activated kinases” according to the invention include kinases that are directly activated by the activation of PRLR and kinases that are activated by the kinases that have been activated by the activation of PRLR. They include in particular JAK kinases such as JAK2, MAP kinase kinase kinases such as Raf kinase, MAP kinase kinases such as MEK1/2, MAP kinases such as ERK1/2, Src kinase, focal adhesion kinase (FAK), phosphoinositide-3 kinase (PI-3K), AKT kinases and p70S6 kinase.

As used herein, the expression “inhibitor of a downstream activated receptor-associated kinase” refers to a compound that prevents the activation of the kinases defined above and/or prevents or decreases the activation of the signaling triggered by said kinases.

The presence of a mutation in a gene can be detected by any of a number of diagnostic assays as defined above in the section “In vitro method of prognosing outcome of hormone-dependent cancer”.

In accordance with one preferred embodiment, the presence of the mutation I146L may be detected as described in Bogorad et al. (2008) Proc. Nat. Acad. Sci. 105:14533-14538 by entirely sequencing the exon 6 of the PRLR gene in both directions using primers matching intronic boundaries, or by performing an ApoI enzymatic digestion on the PCR amplification fragment wherein the mutation is looked for. Similarly, the presence of the mutation I76V may be detected by entirely sequencing the exon 5 of the PRLR gene in both directions using primers matching intronic boundaries, or by restriction with the restriction enzyme HpyCH4 IV.

Nucleic acid samples suitable for performing the detection are the same as those defined in the above section “In vitro method of prognosing outcome of hormone-dependent cancer”.

The expression of a constitutively active mutant of the PRLR may further be confirmed by quantifying the activated form of PRLR in a biological sample previously obtained from the subject as described in the above section “In vitro method of prognosing outcome of hormone-dependent cancer”.

The present invention also relates to a method of treating a hormone-dependent cancer, in particular breast cancer or prostate cancer, and/or preventing progression of a hormone-dependent cancer, in particular breast cancer or prostate cancer, towards a hormone-independent stage, comprising administering a therapeutically effective amount of an inhibitor of PRLR-triggered signaling cascades as defined above in a subject in need thereof, preferably wherein the presence of a constitutively activating mutation in the PRLR gene of said subject has been detected in a nucleic acid sample previously obtained from said subject. Preferably said constitutively activating mutation is a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine. More preferably, said constitutively activating mutation is a mutation resulting in the expression of a mutant prolactin receptor wherein the isoleucine residue at position 146 is substituted with leucine.

The present invention also relates to a method of treating hormone-dependent cancer, in particular breast cancer or prostate cancer, and/or preventing progression of hormone-dependent cancer, in particular breast cancer or prostate cancer, towards a hormone-independent stage, in a subject in need thereof, comprising:

(a) detecting the presence of a constitutively activating mutation in the PRLR gene of said subject in a nucleic acid sample previously obtained from said subject, wherein said constitutively activating mutation is preferably a mutation resulting in the expression of a mutant prolactin receptor wherein the isoleucine residue at position 146 is substituted with a leucine residue or a mutation resulting in the expression of a mutant prolactin receptor wherein the isoleucine residue at position 76 is substituted with valine; and

(b) administering a therapeutically effective amount of an inhibitor of PRLR-triggered signaling cascades as defined above in said subject.

As used herein, the expression “a therapeutically effective amount” of a compound means an amount sufficient to cure, alleviate or partially arrest the clinical manifestations of a given disease.

Preferably, the inhibitors of PRLR-triggered signaling cascades according to the invention defined above are administered in the form of pharmaceutical compositions further comprising at least one pharmaceutically acceptable carrier.

The term “pharmaceutically acceptable carrier” refers to a carrier that may be administered to a patient, together with a compound of this invention, and does not destroy the pharmacological activity thereof and is non-toxic when administered in doses sufficient to deliver a therapeutically effective amount of the compound.

Pharmaceutically acceptable carriers and vehicles that may be used in the compositions of this invention include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As appreciated by skilled artisans, compositions are suitably formulated to be compatible with the intended route of administration. Examples of suitable routes of administration include parenteral route, including for instance intramuscular, subcutaneous, intravenous, intraperitoneal or local intratumoral injections. The oral route can also be used, provided that the composition is in a form suitable for oral administration, able to protect the active principle from the gastric and intestinal enzymes.

The composition according to the invention may be delivered in doses ranging from about 0.01 μg/Kg to about 5000 μg/Kg, alternatively from about 0.1 to about 1000 μg/Kg, alternatively from about 1 to about 500 μg/Kg. Effective doses will also vary depending on route of administration, as well as the possibility of co-usage with other agents.

### An In Vitro Method for Identifying a Subject Suffering from Hormone-Dependent Cancer Likely to Respond to a Treatment with an Inhibitor of PRLR-Triggered Signaling Cascades

The present invention also relates to an in vitro method for identifying a subject suffering from hormone-dependent cancer, in particular breast cancer or prostate cancer, likely to respond to a treatment with an inhibitor of PRLR-triggered signaling cascades as defined above comprising detecting a constitutively activating mutation in the PRLR gene in a nucleic acid sample previously obtained from said subject, said constitutively activating mutation preferably being a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine.

In the context of the invention, the expression “subject suffering from hormone-dependent cancer likely to respond to a treatment” refers to a subject suffering from hormone-dependent cancer which displays statistically better chances to be treated by a particular treatment than another subject suffering from a different type of hormone-dependent cancer.

Inhibitors of PRLR-triggered signaling cascades are as defined above in the section “Inhibitor of PRLR-triggered signaling cascades and uses thereof”.

The presence of a mutation in a gene can be detected by any of a number of diagnostic assays as defined above in the section “In vitro method of prognosing outcome of hormone-dependent cancer”.

In accordance with one preferred embodiment, the presence of the I146L mutation may be detected as described in Bogorad et al. (2008) Proc. Nat. Acad. Sci. 105:14533-14538 by entirely sequencing the exon 6 of the PRLR gene in both directions using primers matching intronic boundaries or by performing an ApoI enzymatic digestion on the PCR amplification fragment wherein the mutation is looked for. Similarly, the presence of the I76V mutation may be detected by entirely sequencing the exon 5 of the PRLR gene in both directions using primers matching intronic boundaries.

Nucleic acid samples suitable for performing the detection are the same as those defined in the above section “In vitro method of prognosing outcome of hormone-dependent cancer”.

The expression of a constitutively active mutant of the PRLR may further be confirmed by quantifying the activated form of PRLR in a biological sample previously obtained from the subject as described in the above section “In vitro method of prognosing outcome of hormone-dependent cancer”.

In a particular embodiment, the above method is for identifying a subject suffering from hormone-dependent cancer, in particular breast cancer or prostate cancer, likely to respond to a treatment with an inhibitor of PRLR-triggered signaling cascades as defined above comprising detecting a constitutively activating mutation in the PRLR gene in a nucleic acid sample previously obtained from said subject, said constitutively activating mutation being preferably a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine; wherein the presence of said constitutively activating mutation in the PRLR gene of said subject indicates that the subject is likely to respond to said treatment with an inhibitor of PRLR-triggered signaling cascades.

In other words, the present invention also relates to an in vitro method for identifying a subject suffering from hormone-dependent cancer, in particular breast cancer or prostate cancer, likely to respond to a treatment with an inhibitor of PRLR-triggered signaling cascades as defined above comprising

(a) detecting a constitutively activating mutation in the PRLR gene in a nucleic acid sample previously obtained from said subject, said constitutively activating mutation preferably being a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine; and

(b) deducing therefrom whether the subject is likely to respond to said treatment with an inhibitor of PRLR-triggered signaling cascades.

The invention will be further illustrated by the following figures and examples.

### DESCRIPTION OF THE FIGURES

FIG. 1 displays pictures showing the morphology of stable clones of parental MCF-7 cells or of MCF-7 cells expressing PRLRWT or PRLRI146L.

FIG. 2 displays pictures showing the progressive evolution along cell passages (early, intermediate, late) of two stable clones of MCF-7 cells expressing PRLRI146L. Circles identify clusters of epithelial cells, arrow identifies examples of individual, mesenchymal-like cells.

FIG. 3 displays histograms representing the expression of typical markers of epithelial versus mesenchymal cells (V: vimentin, L: laminin, Cav1: caveolin 1, Cav2: caveolin 2, S1: Snail1, S2: Snail2, N-Cad: N-Cadherin, E-Cad: E-Cadherin, P-Cad: P-Cadherin, K18: keratin 18, K19: keratin 19, K5: keratin 5, K6: keratin 6, F: fascine), measured by quantitative PCR, expressed in log changes in MCF-7 PRLRI146L cells versus MCF-7 PRLRWT cells. These data are averaged from 3 independent experiments.

FIG. 4 displays pictures of a wound healing assay wherein migration of MCF-7 PRLRWT and MCF-7 PRLRI146L cells was followed up during 48 h after performing a wound in a confluent cell layer. The magnified panel at the bottom shows that while MCF-7 PRLRWT cells were unable to heal upon 48 h, MCF-7 PRLRI146L cells migrated and rapidly filled the wound.

FIG. 5 displays histograms representing the size of a wound (in μm) performed in a confluent cell layer of MCF-7 PRLRWT (WT), of MCF-7 PRLRI146L (I146L) or of SK-BR3 cells (SKBR3) 0 h, 24 h and 48 h after healing. SK-BR3 cells were used as a non migrating cell line. ***: statistically different vs I146L at 0 h. ###: statistically different vs WT at 24 h. §§§: statistically different vs WT at 48 h.

FIG. 6 displays graphics representing proliferation over 4 days of four MCF-7 clones expressing either PRLRWT et MCF-7 PRLRI146L (clones 1, 2 and 3) in growth medium containing 10% FCS and phenol red (two sources of estrogen). Proliferation was measured using WST-1 assay. **: p<0.05 PRLRI146L (3 clones) vs PRLRWT ***: p<0.01 PRLRI146L (3 clones) vs PRLRWT.

FIG. 7 displays histograms representing proliferation of parental MCF-7 cells, MCF-7 cells expressing PRLRWT or MCF-7 expressing PRLRI146L in medium containing no phenol red with 1% charcoal stripped serum, in the absence of estrogen (-E2) or in the presence of 100 nM of estrogen (+E2). Proliferation was measured using WST-1 assay.

FIG. 8 displays the results of Western blots, wherein the expression of the receptors for estrogen (ERa), progesterone (PR) and prolactin (PRLR) was followed up along passages (early to late) in MCF-7 PRLRI146L cells (clones 1 and 3) and MCF-7 PRLRWT cells, using specific antibodies. The expression of tubulin was measured as a control.

FIG. 9 displays graphics representing the expression of HER-2 in MCF-7 PRLRWT cells (black line), in MCF-7 PRLRI146L cells clone 3 (dashed line) and in MCF-7 PRLRI146L cells clone 1 (dotted line) along passages (early to late) using quantitative RT-PCR. The results are presented in relative expression versus early passages of PRLRWT expressing cells.

FIG. 10 displays the unsupervised clustering obtained after analysis of the transcriptomic profiles obtained for the triplicates of the three cell lines (parental MCF-7 cells, MCF-7-PRLRWT and MCF-7-PRLRI146L).

FIG. 11 displays the dendrogram obtained after the following analysis performed on the transcriptomic profiles of FIG. 10. In order to compare these data to previous studies involving expression profile of several breast cancer cell lines (Neve et al. (2006) Cancer Cell 10:515-527; Charafe-Jauffret et al. (2006) Oncogene 25:2273-2284), expression values were transformed to −1, 0 or 1 depending on their position compared to the median of their distribution. When expression value was within the ±10% range of the median, it was replaced by 0; below 10%, it was replaced by −1, and above 10%, by +1. Unsupervised clustering of these new matrices was then performed. The dendrogram obtained after this analysis confirmed the existence of two distinct groups, corresponding to so-called “basal” and “luminal” cell lines. The position of parental MCF-7 cells (parental), MCF-7 PRLRWT cells (PRLR WT) and MCF-7 PRLRI146L cells (PRLR I146L) is indicated on the dendrogram.

FIG. 12 displays graphics representing the percentage of tumor-free mice, during 50 day follow up after injection of MCF-7 PRLR WT xenografts (black line) or MCF-7 PRLR I146L xenografts (dashed lines) in nude mice, in the absence of estradiol supply.

FIG. 13 displays graphics representing the weight of tumors (in mg) at resection after injection of MCF-7 parental xenografts (MCF-7 par), MCF-7 PRLR WT xenografts (MCF-7 PRLR WT), MCF-7 PRLR I146L xenografts (MCF-7 PRLR I146L) or MDA-MB-231 xenografts (MDA-MB-231) in nude mice, in the absence of estradiol supply.

FIG. 14 displays pictures showing the invasiveness properties of MCF-7 PRLRI146L xenografts in derma (left panel) and muscle (right panel).

FIG. 15 displays pictures showing the presence of pulmonary micro-metastases (arrows) 15 days after tumor resection in the groups of MCF-7 PRLR WT xenografts (MCF-7 PRLR WT), MCF-7 PRLR I146L xenografts (MCF-7 PRLR I146L) or MDA-MB-231 xenografts (MDA-MB-231).

FIG. 16 displays histograms representing the number of micro-metastases counted in 2 lungs of mice 15 days after tumor resection in the groups of MCF-7 PRLR WT xenografts (MCF-7 PRLR WT), MCF-7 PRLR I146L xenografts (MCF-7 PRLR I146L) or MDA-MB-231 xenografts (MDA-MB-231) or in control mice with only matrigel injection (control).

FIG. 17 displays the results of FACS analysis of CD24 and CD44 markers in SKBR3, MDA-MB-231, and MCF-7 PRLR WT and I146L cells.

FIG. 18 displays the results of FACS analysis of FACS analysis of MDA-MB-231, and MCF-7 PRLR WT and I146L cells using the ALDEFLUOR assay. Cells incubated with the specific ALDH inhibitor (DEAB, upper panel), were used to determine the baseline fluorescence of these cells. Incubation of cells with ALDEFLUOR substrate BAAA in the absence of DEAB (ALDH, lower panel) induces a shift in BAAA fluorescence (arrow) defining the ALDEFLUOR-positive population.

### EXAMPLE

The following example presents results that support the role of constitutive PRLR signalling, exemplified by PRLR-I146L variant, in progression of established breast cancer.

### Material and Methods

### Cell Cultures and Transfections

MCF-7 cells were cultured in DMEM, 10% FCS, 2 mM Glutamine and antibiotics. Cells were co-transfected (Fugene 6, Roche) using plasmids encoding the human PRLR, WT or I146L, as described in Bogorad et al (2008). Proc. Natl. Acad. Sci U.S. A 105:14533-14538. Stable clones were selected in growth medium containing 500 μg/mL active G-418 (geneticin). Three clones of MCF-7-PRLR_{I146L} cells (clones 1, 2 and 3) were selected for comparative analysis with MCF-7-PRLR_{WT} cells and parental MCF-7 cells (non transfected).

### Quantitative RT-PCR

Total RNA were isolated from cells using the QIAGEN RNeasy kit (Valencia, Calif., USA) according to manufacturer's instructions. RNA (250 ng) was reverse transcribed using SuperScript™II Reverse transcriptase using the SuperScript™II First-Strand Synthesis System for RT-PCR kit (Invitrogen, CA, USA). For Q RT-PCR analysis, the cDNA was then subjected to real-time PCR amplification using gene specific primers and SYBR® Green PCR Master Mix (Applied Biosystems). Primers sequences are provided in table 1 below and each primer was used at a 250 nM final concentration. Cyclophilin B was employed as a housekeeping gene in each reaction. Real-time PCR was performed on a “7300 Fast real time PCR system” (Applied Biosystems, Paris, France). Results were obtained with the Applied Biosystems 7300 software. Data were analyzed by the comparative cycle threshold method and presented as fold change in gene expression relative to internal calibrators as mentioned in the figures. Three independent experiments (from three different sets of RNA preparation) were performed in duplicate and the results were expressed as means±S.D.

TABLE 1
Primers sequences used in the example

| primer | sequence | SEQ ID |
|---|---|---|
| N-cadherin-F | AAATTGAGCCTGAAGCCAAC | 5 |
| N-cadherin-R | GTGGCCACTGTGCTTACTGA | 6 |
| E-cadherin-F | CCTGGGACTCCACCTACAGA | 7 |
| E-cadherin-R | TGTGAGCAATTCTGCTTGGA | 8 |
| P-cadherin-F | GGAGACAGGCTGGTTGTTGT | 9 |
| P-cadherin-R | CCACTGAGGCACCATTCTCT | 10 |
| KRT18-F | CACAGTCTGCTGAGGTTGGA | 11 |
| KRT18-R | CAAGCTGGCCTTCAGATTTC | 12 |
| KRT19-F | ACTACACGACCATCCAGGAC | 13 |
| KRT19-R | TCTCAAACTTGGTTCGGAAG | 14 |
| Vimentin-F | TGCCTTTTTGAAGAAACTCCA | 15 |
| Vimentin-R | GTGAGGTCAGGCTTGGAAAC | 16 |
| Laminin-F | GCCACCATCTACCTCCTCAA | 17 |
| Laminin-R | CAGACAGAAGGCTTTTCATCG | 18 |
| Fascin-F | TCCAATGGCAAGTTTGTGAC | 19 |
| Fascin-R | GATGATGGGGCGGTTGAT | 20 |
| Caveolin 1-F | GCGACCCTAAACACCTCAAC | 21 |
| Caveolin 1-R | ACAGCAAGCGGTAAAACCAG | 22 |
| Caveolin 2-F | GGCTCAACTCGCATCTCAAG | 23 |
| Caveolin 2-R | TGATTTCAAAGAGGGCATGG | 24 |
| hTwist1-F | GTCCGCAGTCTTACGAGGAG | 25 |
| hTwist1-R | GCAGCGTGGGGATGATCT | 26 |
| hSNAIL1-F | CTCTTTCCTCGTCAGGAAGC | 27 |
| hSNAIL1-R | GTCGTAGGGCTGCTGGAAG | 28 |
| hSNAIL2-F | ATTTCAACGCCTCCAAAAAG | 29 |
| hSNAIL2-R | TTGTGGTATGACAGGCATGG | 30 |
| hPR-F | TCCTTACCTGTGGGAGCTGT | 31 |
| hPR-R | GCGGATTTTATCAACGATGC | 32 |
| hERα-F | TGGAGTCTGGTCCTGTGAGG | 33 |
| hERα-R | GGCAGCTCTTCCTCCTGTTT | 34 |
| HER2-F | TGTGACTGCCTGTCCCTACA | 35 |
| HER2-R | GCTTGCTGCACTTCTCACAC | 36 |
| hPRLR-F | CTGGGACAGATGGAGGACTT | 37 |
| hPRLR-R | GGGCCACCGGTTATGTAGT | 38 |
| Cyclophilin-F | CAGGTCCTGGCATCTTGTCC | 39 |
| Cyclophilin-R | TTGCTGGTCTTGCCATTCCT | 40 |

### Transcriptomic Analysis (Affymetrix)

Total RNA were purified with the QIAGEN RNeasy kit (Valencia, Calif., USA) and quality was assessed on the Agilent BioAnalyzer (all RINs were 10). Preparation of in vitro transcription (IVT) products, oligonucleotide array hybridization, and scanning were performed according to Affymetrix (Santa Clara, Calif.) protocols. In brief, 100 ng of total RNA from each cell line and T7-linked oligo-dT primers were used for first-strand cDNA synthesis. IVT reactions were performed to amplify biotinylated cRNA targets, which were purified and assessed for their quality on a Agilent BioAnalyzer before chemical fragmentation at 95° C. for 35 min. Fragmented biotinylated cRNA (12.5 μg) was hybridized at 45° C. for 16 h to Affymetrix GeneChip Human Genome U133 Plus 2.0 Array. The arrays were then washed and stained with streptavidin-phycoerythrin (SAPE; final concentration 10 mg/ml). Signal amplification was performed using a biotinylated anti-streptavidin antibody. The array was scanned according to the manufacturer's instructions (Scanner 7G). Scanned images were inspected for the presence of obvious defects (artifacts or scratches) on the array.

### Wound Healing Assay

MCF-7 PRLR_{WT} cells and MCF-7 PRLR_{I146L} clone 1 were cultured at near 90% confluence, in normal growth medium, in 6 well dishes marked with a line on the bottom of the dish. On the day of the assay, for each cell line, three separate scratches were made through the cells, perpendicular to the line drawn, using a sterile 200 μl pipet tip. Cell medium was aspirated, cells were rinsed with PBS, and the dish was filled with medium. Pictures of cells were made with a phase contrast microscope (Nikon Eclipse Ti) at 10× magnification at various times. Wound filling was assessed at 24 and 48 h. At each time, the medium was replaced; pictures were taken to enable measuring the size of the wound (Imaging software NIS Element F 3.0, Nikon). Pictures shown are representative of five experiments performed independently and measurements are shown on histogram graphs as means±S.D. For each scratch measurement, six measures were performed in three independent pictures.

### Modified Boyden Chamber Assay for Cell Invasion Assessment

Cell invasion assays were performed in an Invasion Chamber with 8 mm pore size polycarbonate membrane inserts for 24-well plates (ECM 550, Chemicon International, Millipore). Filters were coated with a dried ECMatrix™, a reconstituted basement membrane matrix of proteins derived from the Engelbreth Holm-Swarm (EHS) mouse tumor. Cell suspensions containing 750,000 cells/ml were added to the upper chamber in 300 μl of serum-free medium and incubated for 24-48 h. The lower chamber was filled with 500 μl of serum containing 10% SVF medium. After incubation, non-invading cells and ECMatrix gel from the interior of the insets were removed using cotton-tipped swabs, and invasive cells on the underside of the filter were stained with 0.5% toluidine blue for 20 min, rinsed with water and dried. Macroscopic pictures of the inserts were made on a microscope. The assay was performed 5 times for each clone.

### Western Blotting

Cell lysates were prepared as described in Llovera et al. (2000) Oncogene 19:4695-4705. Fifty μL of total lysates were loaded on SDS-PAGE and transferred to nitrocellulose membrane for immunoblotting analysis. Antibodies were as follows: anti-human PRLR (clone 1A2B1, Zymed-Invitrogen, 1:1,000 dilution), estradiol receptor (ER) (Santa Cruz, sc8002, 1:200 dilution), progesterone receptors isoforms A and B (PR-A, Pr-B) (Santa Cruz, sc539, 1:500 dilution), and tubulin used as loading control (Sigma, T9026 1:1000 dilution). Antigen-antibody complexes were revealed using either horseradish peroxidase conjugated with anti-mouse (1:4,000 dilution) or with anti-rabbit (1:10,000 dilution) antibody (GE Healthcare).

### Cell Proliferation

Cell proliferation/survival was measured over 2 to 3 days by daily measurement of tetrazolium salt conversion (WST-1 assay), as described in Bogorad et al. (2008) Proc. Natl. Acad. Sci. U.S.A 105:14533-14538. Briefly, cells were trypsinized, counted and distributed in 96 well plates at a density of 50,000 cells per well. 10 μL WST-1 was added and optical density at 450 nm (OD450) was measured using a multiplate reader (Mithras, Berthold). For experiments involving investigation of estradiol effect, the inventors used RPMI medium 1640 without phenol red (Gibco, Invitrogen) and charcoal stripped fetal bovine serum (CSS) purchased from Gibco, Invitrogen. Estradiol was provided by Sigma and was dissolved in EtOH (same EtOH concentration was added in all wells).

### FACS Analysis (CD24/CD44)

The anti-CD44 (FITC Mouse Anti-Human, clone G44-26) and anti-CD24 (PE Mouse Anti-Human, clone ML5) antibodies used for FACS analysis were obtained from BD Bioscience. SYTOX® Blue (Invitrogen) was used for live/dead cell distinction. For low cytometric analysis, cells were detached with Versene 2 mM at 37° C., then washed, resuspended in PBS-1% BSA before counting. One million cells were used for each conditions. Cells were incubated 1 h, at 4° C. with 20 μl of either anti-CD24 or anti-CD44 antibodies for simple staining or 20 μl for both antibodies for dual staining. Cells were then washed and resuspended in cold PBS. Analysis was performed on a BD LSRF FORTESSA SORP FACS (BD Biosciences) and data were treated with the BD FACSDiva Software (BD Biosciences). Assay was performed 3 times.

### ALDEFLUOR Assay

The ALDEFLUOR kit purchased from StemCell Technologies was used to determine the aldehyde dehydrogenase (ALDH) enzymatic activity using a BD LSRF FORTESSA SORP FACS (BD Biosciences). Briefly, cells were incubated in ALDEFLUOR assay buffer containing ALDH substrate (BAAA) (1 μmol/L per 10⁶ cells). In each experiment, a sample of cells was stained under identical conditions with 50 mmol/L of diethylaminobenzaldehyde (DEAB), a specific ALDH inhibitor, as negative control. The sorting gates were established using SYTOX® Blue (Invitrogen) for live/dead cell distinction. Shift in mean fluorescence intensity (MFI) was calculated as the ratio between MFI in DEAB condition vs MFI in BAAA condition. Assay was performed in triplicate.

### Xenografts

One week before cell injection, one 60 day pellet of 1713-estradiol was subcutaneously implanted in subgroups (named E2+) of Nude mice of 3 months of age (purchased from Janvier). Other subgroups did not receive this treatment (named E2−). One week later, cells to be injected (MCF-7 PRLR_{WT}, MCF-7 PRLR_{I146L} cells, MDA-MB231 cells) were mixed with PBS/Matrigel® at a final concentration of 10⁸ cells/mL, and 50 μL (5×10⁶ cells) were injected into the mammary fat pad of the 4^{th} mammary gland, unilaterally. Control mice received only PBS/matrigel (no cell). Mice were followed up twice a week (general state, weight, measurement of tumor volume). Tumor volume was estimated by measuring tumor length (d1) and width (d2), as follows: V=(d1×d2²)/2. It was expressed in mm³. The experiment lasted until tumors achieved (whenever they did) the ethically acceptable maximal volume of 2,500 mm³. Practically, tumors were harvested after 74 days, except for the group E2+/MCF-7 PRLR_{I146L} cells in which primary tumors were removed at day 61. In all groups, some mice were kept alive after tumor resection for 2 or 4 additional weeks in order to evaluate tumor recurrence and metastatic potential. Various tissues (lungs, lymph nodes, bones) were harvested at sacrifice to evaluate/quantify the occurrence of metastases. Histological analyses of tumor and tissues were performed by pathologists.

### Results

In order to investigate the effects of constitutive PRLR signalling in a mammary context, the inventors generated various stable clones of MCF-7 breast cancer cells stably expressing this variant (called MCF-7 PRLR_{I146L} cells). Clone 1 was the one that was used in the publication of Bogorad et al. (Bogorad et al. (2008) Proc. Natl. Acad. Sci U.S.A 105:14533-14538) to demonstrate constitutive signalling and mitogenic potency of PRLR-I146L. Clones 2 and 3 were generated in the same experiment, but were never used before. The three clones were compared in all experiments to the parental MCF-7 cell line, and to a stable clone transfected using a vector encoding the WT hPRLR (MCF-7 PRLR_{WT} cells). It is noteworthy that MCF-7 PRLR_{I146L} cells are an interesting model as they express both wild type PRLR (endogenous) and PRLR-I146L (transfected), thereby reconstituting the “heterozygous” context observed in the patients identified so far to harbour the PRLR-I146L allele (Canbay et al. (2004) Curr. Med. Res. Opin. 20:533-540; Courtillot et al. (2010) J. Clin. Endocrinol. Metab 95:271-279).

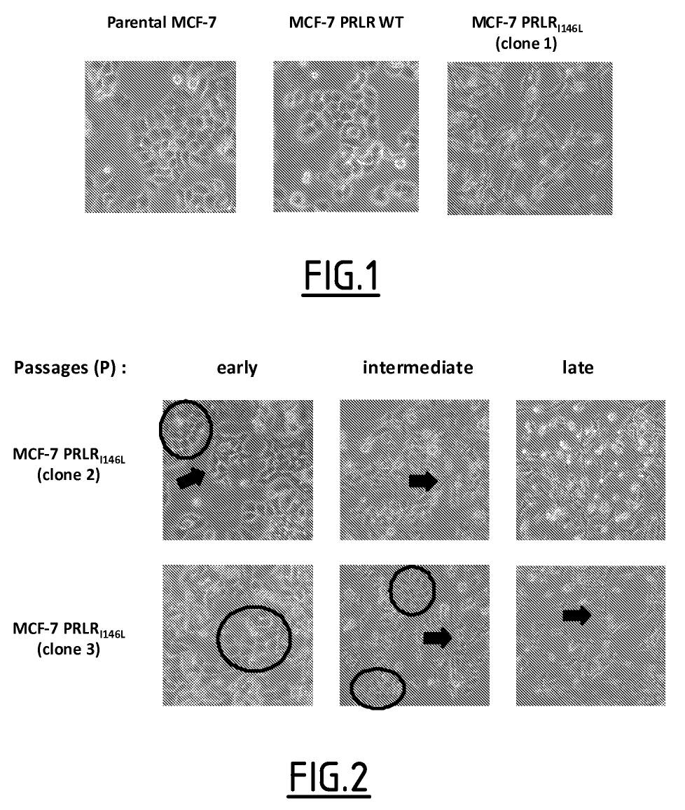

As shown in FIG. 1, MCF-7 PRLR_{WT} cells displayed similar morphology compared to parental cell line, indicating that expression of PRLR-WT per se does not exert any detectable effect. In contrast, MCF-7 PRLR_{I146L} cells displayed a very different morphology, as they resembled fibroblasts or mesenchymal cells. The progressive morphological transition was clearly highlighted by following-up clones 2 and 3. As shown on FIG. 2, clusters of epithelial cells (circles) are still detected at early passages, but cells rapidly evolve with time (along passages) to the mesenchymal/fibroblast (arrows) morphology. The fact that the three clones underwent the same morphological changes support that the latter are actually induced by PRLR-I146L.

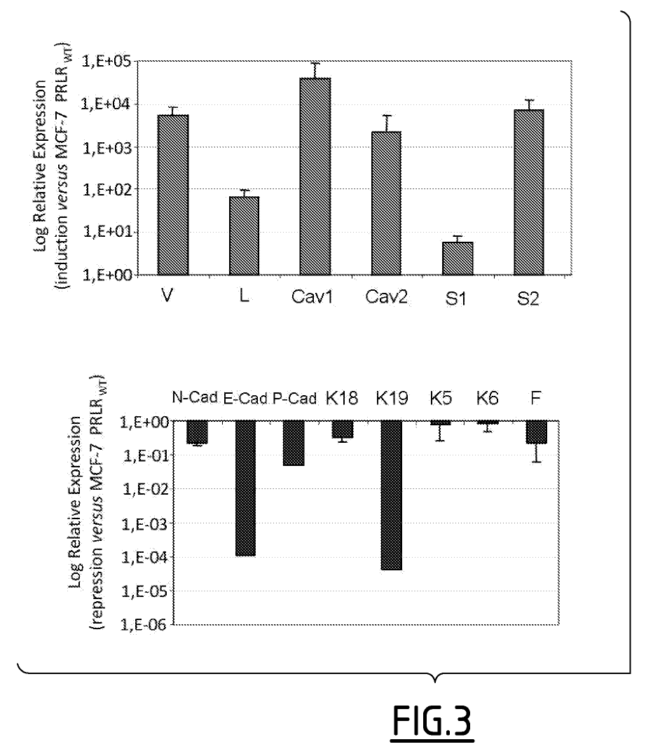

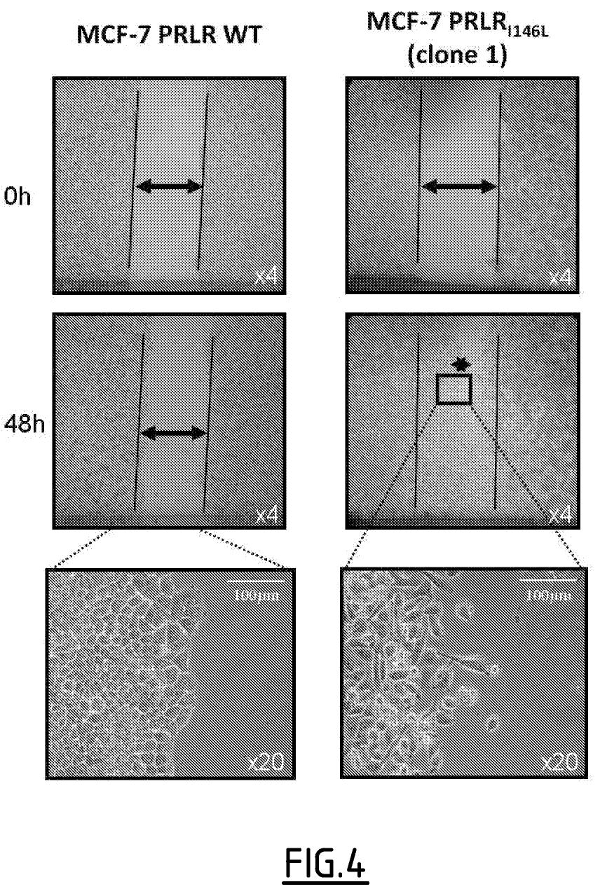

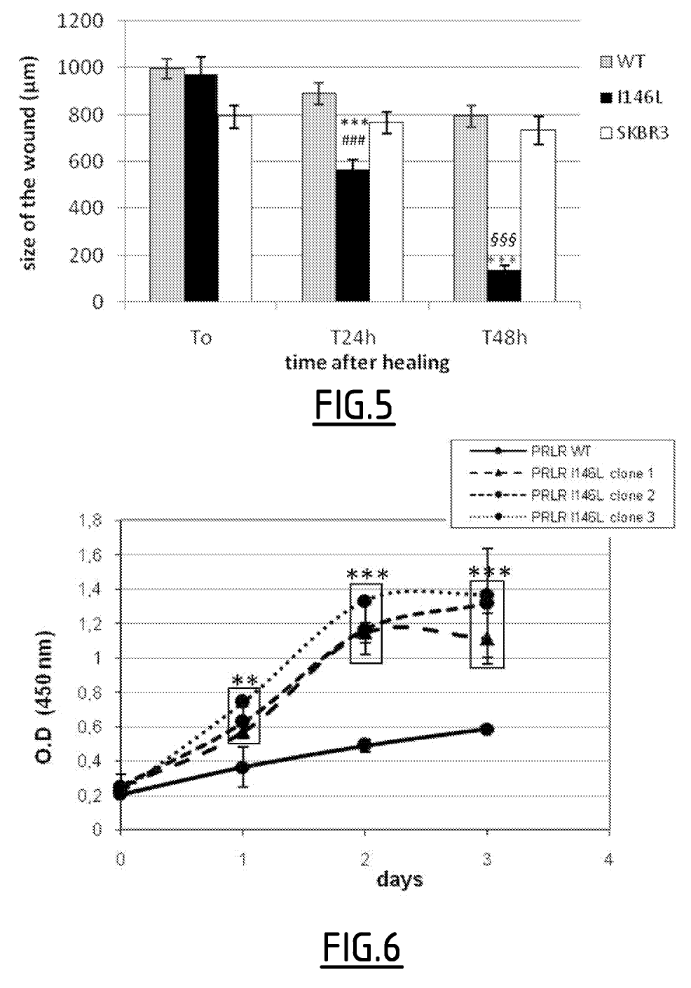

These morphological changes are reminiscent of a well known process termed epithelial-to-mesenchymal transition, or EMT. This is a key process normally occurring during development of multicellular organisms. In cancer, it is assumed to participate in the metastatic process as EMT disrupts cell-cell contacts, and favours cell migration and invasion. EMT can be characterized by a series of well accepted markers and by the acquisition of new biological properties. The inventors measured the more classical EMT markers using Q-RT-PCR. As shown on FIG. 3, MCF-7 PRLR_{I146L} cells lost several epithelial markers (K18/19, cytokeratin 18/19; E-cad, E-cadherin; F, Fascin), and gained typical mesenchymal markers (V, vimentin; L, laminin; S1/S2, Snail 1/2). Regarding functional properties, FIG. 4 shows the classical wound healing assay that is used to demonstrate the ability of epithelial cells to migrate on a plastic support. While MCF-7 PRLR_{WT} failed to migrate and to wound heal (FIG. 4), MCF-7 PRLR_{I146L} migrated and rapidly filled the wound (see magnified panel at the bottom). The size of the wound was measured through 2 day follow-up, using SKBR-3 as a negative control of cell migration (FIG. 5). Finally, the inventors used the Boyden chamber assay to ascertain the ability of cells to invade a biological membrane mimicking the extracellular matrix normally surrounding cells in vivo. According to the wound healing assay, MCF-7 PRLR_{I146L} cells migrated and penetrated the matrix while MCF-7 PRLR_{WT} did not. Taken together, these data confirm that expression of PRLR_{I146L} triggers the EMT program in MCF-7 cells.

The inventors next measured in vitro cell proliferation of the various clones. As shown on FIG. 6, MCF-7 PRLR_{I146L} cells proliferated at higher rate compared to MCF-7 PRLR_{WT} cells in growth medium, and this was true for the three clones analyzed.

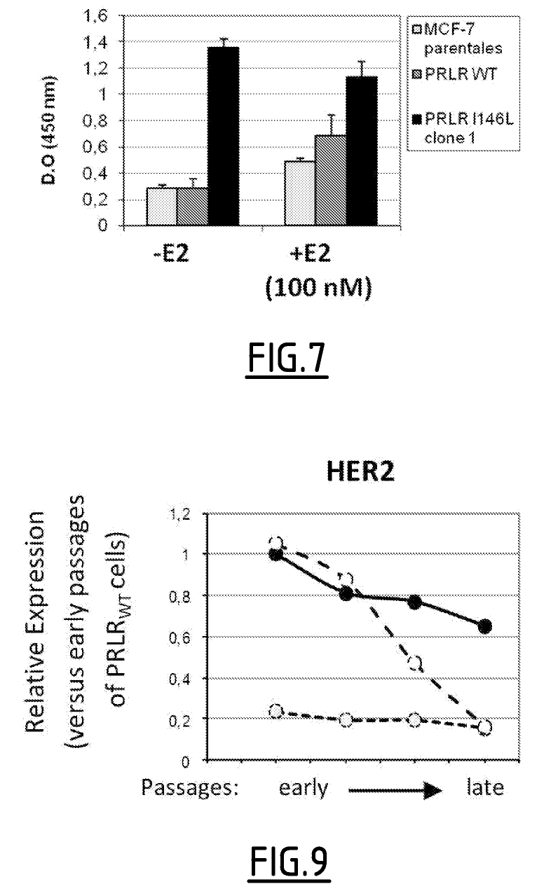

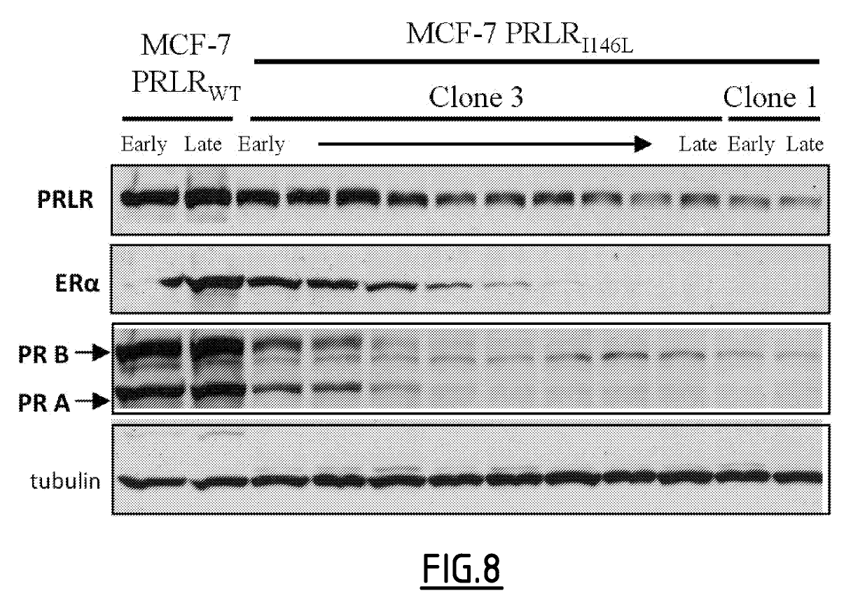

Interestingly, this property was maintained when cells were maintained in low serum, estrogen-free medium (FIG. 7). Even more intriguing, while MCF-7 PRLR_{WT} cells are known to be estrogen-responsive, MCF-7 PRLR_{I146L} cells were found to have lost estrogen-responsiveness (FIG. 7). At the molecular level, these findings were supported by the loss of the Estrogen receptor alpha (ERα). This feature occurred progressively along passages (i.e. during EMT process), which was clearly evidenced when analyzing MCF-7 PRLR_{I146L} clone 3 (FIGS. 8 and 9). Progesterone and prolactin receptors, two other hallmarks of differentiated mammary cells, were also lost/decreased simultaneously (FIG. 8). The level of hormonal receptors did not change along passages in the positive control (MCF-7 PRLR_{WT} cells), indicating that passages per se (=culture aging) were not responsible for these changes in hormone receptor expression. The latter was very low from early passages in MCF-7 PRLR_{I146L} cells clone 1, according to the observation that this clone had already evolved though EMT when it was used for this study (see FIG. 1). The data related to hormone receptor expression at the protein level were confirmed at the mRNA level using Q-RT-PCR for the 3 receptors; this approach also showed that HER-2 (erbb2), another receptor of interest for characterizing breast cancer cells, was also down-regulated in MCF-7 PRLR_{I146L} cells (FIG. 9). Together, these observations indicate that PRLR_{I146L} expression switches MCF-7 cells to functional hormone-independence, according to the loss of hormone receptor expression.

Further, the inventors have found that two breast cancer cell lines, named SKBR-3 and MDA-MB436, previously disclosed for instance by Lacroix and Leclerq (Breast cancer Research and Treatment, (83): 249-289, 2004) and Cailleau et al. (In vitro, 14: 911-915, 1978), “naturally” harbor one PRLR_{I146L} allele.

SKBR-3 cell line does not express estrogen receptors and progesterone receptors and over-expresses HER2 (exhibiting the EP−/ER−/HER2+ profile), whereas MDA-MB436 cell line does not express estrogen receptors and progesterone receptors, and expresses a normal amount of HER2 (exhibiting the triple-negative profile ER−/PR−/HER2−).

These data confirm that expression of a constitutively activated prolactine receptor, for instance PRLR_{I146L}, can trigger loss of expression of hormone-receptors, in particular estrogen and progesterone receptors.

The loss of hormonal receptors (ER, PR), associated with under-expression of HER-2, and the acquisition of hormone-independent proliferation is typical of a category of breast cancers referred to as “triple negative” (i.e. negative for the above mentioned receptors). These cancers are known to be very aggressive and highly metastatic. In addition, they can no longer be targeted using anti-hormonal treatments (e.g. tamoxifen, herceptin) as they do not express anymore the cognate receptors. The features of triple negative breast cancer shares common points with so-called basal-like breast cancers, which can be identified based on their molecular (transcriptomic) signature (Bertucci et al. (2009) Int. J. Cancer 124:1338-1348). To further characterize the phenotypic changes induced by PRLR_{I146L} when expressed in MCF-7 cells, the inventors analyzed the transcriptomic profiles of MCF-7 PRLR_{WT} and MCF-7 PRLR_{I146L} cells clones. They performed study using wide-genome microarrays (Affymetrix).

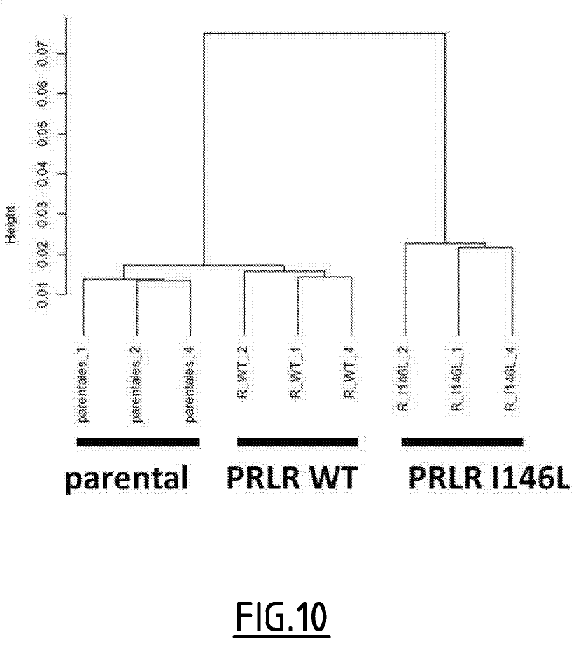

Unsupervised analysis of their transcriptomic profiles showed that parental and MCF-7-PRLR_{WT} cells had very similar profiles, as only 121 probes out of 55.000 presented were found to be differentially regulated (>1.5 fold) (FIG. 10). This suggested that over-expression of the WT receptor per se does not induce drastic changes in gene expression. In sharp contrast, MCF-7-PRLR_{I146L} cells differed from parental and MCF-7-PRLR_{WT} cells by 3,943 or 3,789 probes respectively, therefore they spontaneously clustered in a different group. Several genes known to participate in EMT were highly over-expressed in MCF-7-PRLR_{I146L} cells, confirming the phenotypic changes described above. Two independent studies (Charafe-Jauffret et al. (2006) Oncogene 25:2273-2284; Neve et al. (2006) Cancer Cell 10, 515-527) recently reported the transcriptomic profiles of several breast cancer cell lines (31 and 51 respectively). Global hierarchical clustering discriminated two groups of breast cancer cell lines: group 1 corresponded to luminal cells (well differentiated and expressing ER/PR), whereas group 2 corresponded to basal and mesenchymal cells (poorly differentiated, highly invasive and metastatic in vivo).

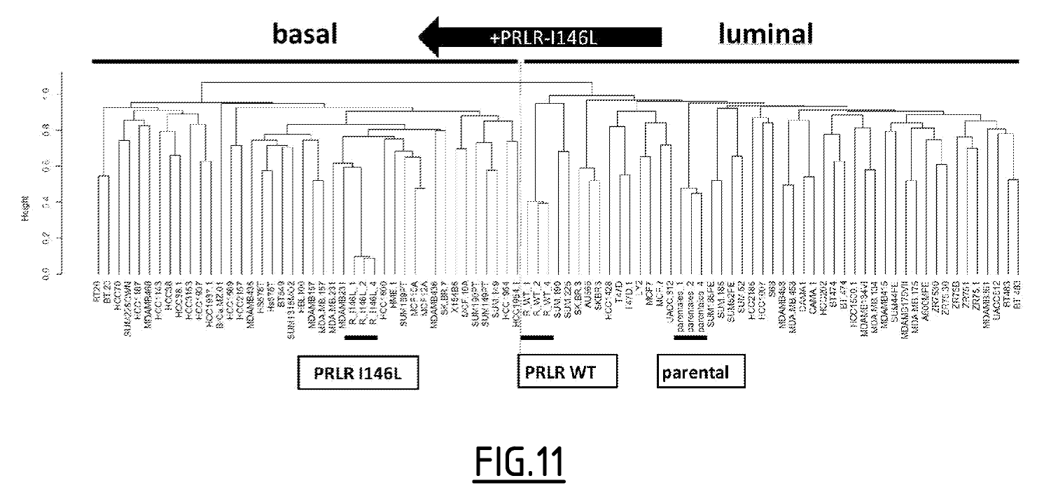

Unsupervised meta-analysis of the present data merged with the two above-mentioned datasets showed that MCF-7-PRLR_{I146L} cells naturally segregated in the basal group, whereas parental and MCF-7-PRLR_{WT} cells expectedly clustered with luminal cells (FIG. 11). Basal-like breast cancers can be subdivided in Basal A and Basal B (or mesenchymal) groups (Charafe-Jauffret et al. (2006) Oncogene 25:2273-2284; Neve et al. (2006) Cancer Cell 10, 515-527). MCF-7-PRLR_{I146L} cells clustered within basal B/mesenchymal group. These findings indicate that constitutive PRLR signaling induced by PRLR_{I146L} shifts luminal breast cancer cells (MCF-7) to a basal B-mesenchymal transcriptomic signature, which defines the most aggressive breast cancers.

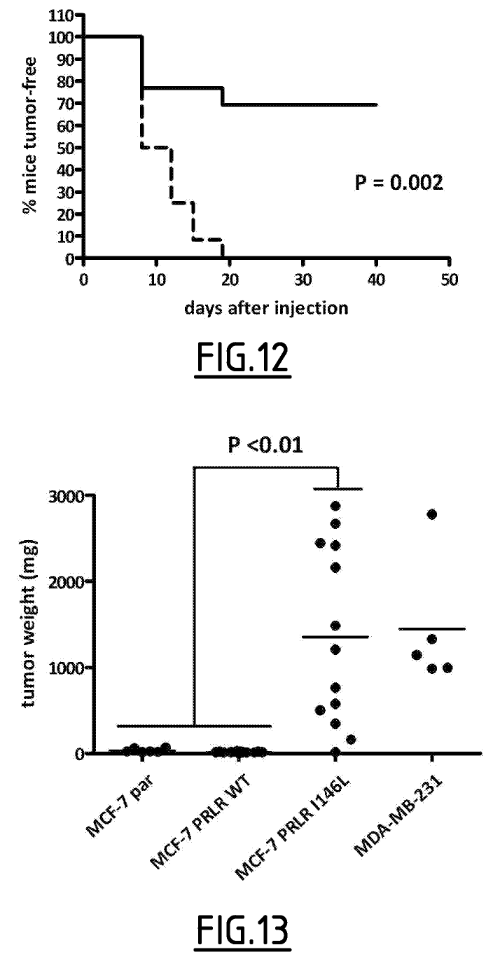

As all dedifferentiated, basal B breast cancer cells, MCF-7-PRLR_{I146L} are invasive and aggressive in vitro. The inventors assessed these properties in vivo using a model of xenograft in immunodeficient mice. The growth of MCF-7 cells (parental or MCF-7-PRLR_{WT}) in mice requires supplementation with estradiol pellets; in the absence thereof, both tumor take and tumor growth, if any, are very limited (FIGS. 12 and 13). In contrast, 100% tumor take was observed for MCF-7-PRLR_{I146L} cells in the absence of estradiol treatment. In addition, tumor weight at resection was dramatically higher compared to MCF-7-PRLR_{WT}, and was actually similar to that of MDA-MB231, a basal-like, triple negative cell line known for its aggressiveness in vivo. This confirms that MCF-7-PRLR_{I146L} cells acquired hormone-independence capacity to grow in vivo.

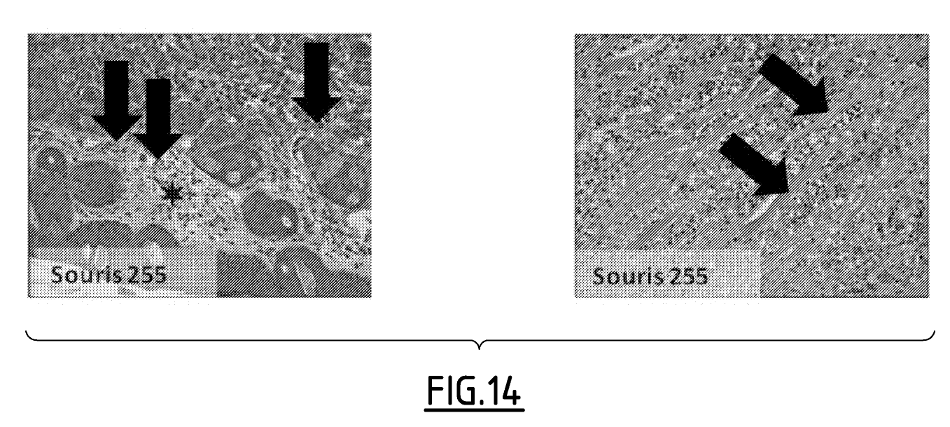

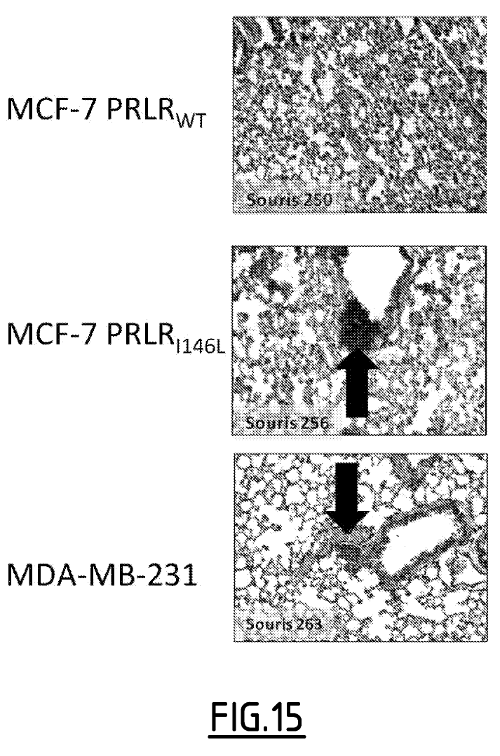

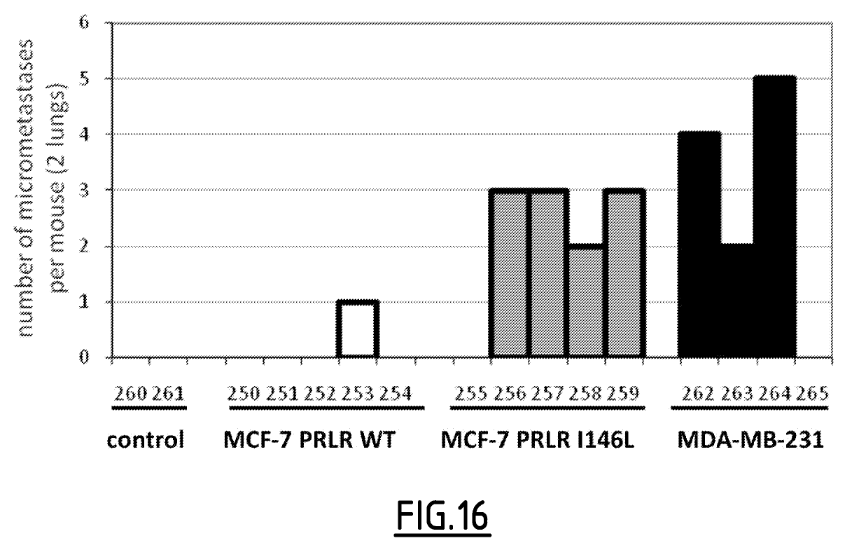

The metastatic potential of MCF-7-PRLR_{I146L} cells was evaluated by local invasion of primary tumors in their environment (mammary gland, muscle, derma) (FIG. 14) and by the occurrence of micrometastases in the lungs 2 weeks after removal of the primary tumors (FIGS. 15 and 16). While no invasion and one micrometastasis was marginally observed in MCF-7-PRLR_{WT} xenografts, these features were often observed for MCF-7-PRLR_{I146L} xenografts, similarly to what was observed for the aggressive MDA-MB231 xenografts. Similar analysis was performed 4 weeks after resection of the primary tumor, when tumor recurrence was observed in some animals (Table 2).

TABLE 2
Tumor recurrence and metastases 4 weeks after tumor resection

| | PRLR WT | | PRLR I146L | | MDA MB |
|---|---|---|---|---|---|
| | −E2 | +E2 | −E2 | +E2 | 231 −E2 |
| Tumor recurrence | 0/12 | 1/8 | 4/13 | 7/9 | 3/4 |
| Weight of tumor | — | 4 mg | 58-786 mg | 8-2737 mg | 11-59 mg |
| Local | — | — | 3/4 | 3/7 | 1/3 |
| invasiveness | | | | | |
| Macrometastases | | | | | |
| Lymph node | 0/12 | 0/8 | 3/13 | 3/9 | 0/4 |
| Lung | 1/6 | 0/6 | 1/5 | 2/6 | 2/5 |
| | Small, | | Diffuse | Diffuse | Small, |
| | localized | | | and big | localized |

Again, while MCF-7-PRLR_{WT} xenografts occasionally displayed recurrence of a very small tumor in one animal or a small and localized pulmonary metastasis in another, tumor recurrence was frequent in MCF-7-PRLR_{I146L} xenografts which also exhibited invasion (macrometastases) in lymph nodes and lungs. The weights of recurrent tumors were sometimes as big as the primary tumor itself, reflecting a high proliferation capacity of cells remaining in the mammary gland after resection of the primary tumor(s) (Table 2). These data confirm that MCF-7-PRLR_{I146L} cells are highly aggressive and have the capacity to disseminate in various organs.

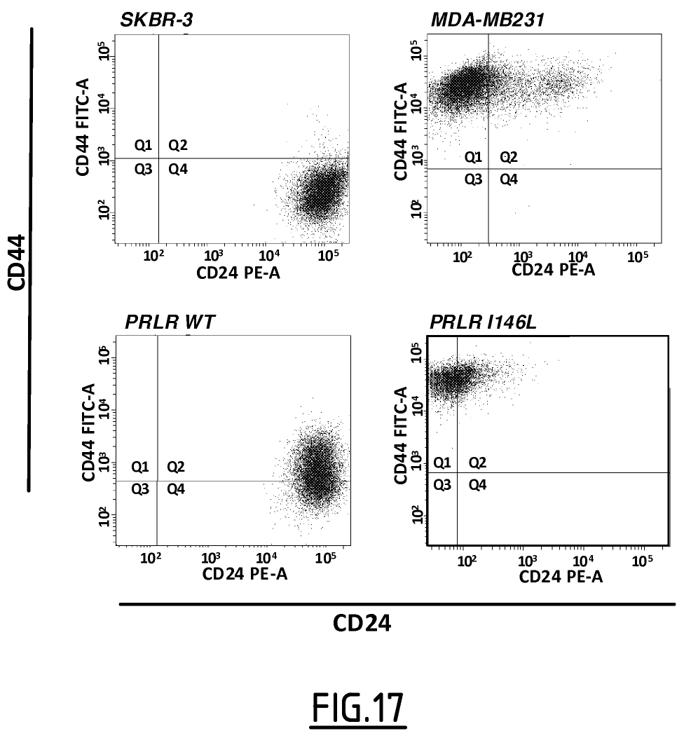

It has been recently proposed (Mani et al. (2008) Cell 133:704-715) that EMT results in the acquisition of stem-cell markers and properties. The inventors investigated two of these features in their clones. First, they analyzed the expression of CD24/CD44 surface markers that have been proposed to be typical of mammary stem-like cells. As shown on FIG. 17 and Table 3, MCF-7-PRLR_{WT} cells exhibited CD24^{low}/CD44^{high} profile typical of differentiated epithelial cells, e.g. SKBR-3 breast cancer cells. MCF-7-PRLR_{I146L} exhibited the reverse profile: CD24^{high}/CD44^{low}.

TABLE 3
FACS analysis of CD24 and CD44 markers in SKBR3, MDA-MB-231
and MCF-7 PRLR WT and I146L cells

| | CD44 | | CD24 | |
|---|---|---|---|---|
| SKBR3 | 0% | | 82.9% | (±14%) |
| MDA-MB-231 | 80% | (±5%) | 20% | (±4%) |
| MCF-7 WT | 0% | | 60.5% | (±5%) |
| MCF-7 I146L | 84.9% | (±9%) | 0% | |

Percentages of cells expressing CD24 and CD44 are expressed as mean ± standard deviation of three independent experiments.

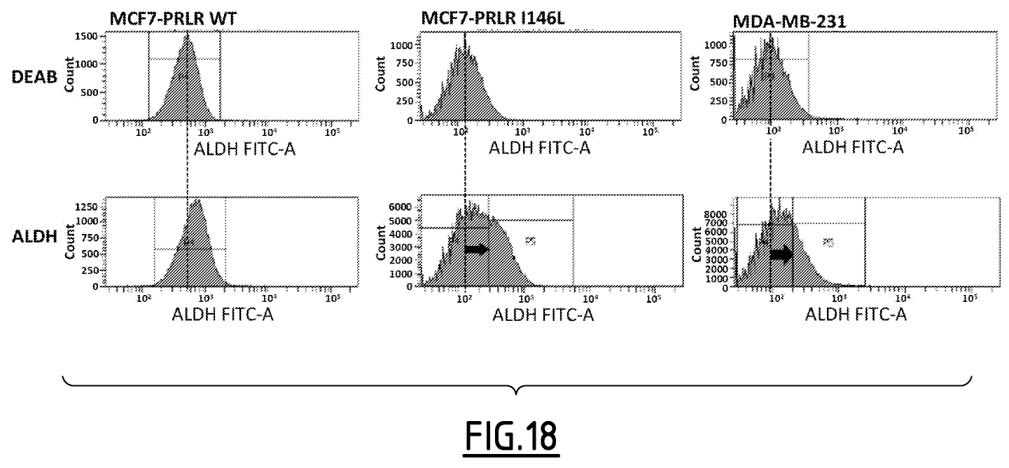

In addition, increased aldehyde dehydrogenase (ALDH) activity as assessed by the ALDEFLUOR assay has been proposed to identify cancer stem cells exhibiting high proliferative and metastatic potential (Charafe-Jauffret et al. (2010) Clin. Cancer Res. 16:45-55; Charafe-Jauffret et al. (2009) Cancer Res. 69:1302-1313). The inventors used this assay to characterize their cells. When Aldefluor activity was compared in the presence or the absence of DEAB inhibitor (used to achieve activity baseline), there was no difference for MCF-7-PRLR_{WT} cells, indicating no intrinsic ALDH activity. In contrast, a marked shift was observed for MCF-7-PRLR_{I146L} cells, similar to that of MDA-MB231 cells, indicating constitutive activation of ALDH (FIG. 18 and Table 4).

TABLE 4
FACS analysis of MDA-MB-231 and MCF-7 PRLR WT and I146L
cells using the ALDEFLUOR assay.

| | DEAB | ALDH | MFI ratio |
|---|---|---|---|
| PRLR WT | 519 | 728 | 1.40 |
| PRLR I146L | 147 | 512 | 3.48 |
| MDA-MB-231 | 110 | 350 | 3.18 |

Mean Fluorescence Intensity (MFI) was defined at the peak of fluorescence in each experiment and the shift of fluorescence was calculated as the ratio between MFI ALDH and MFI BAAA.

In addition to reflect one more drastic phenotypic change induced by PRLR_{I146L}, this observation suggests that MCF-7-PRLR_{I146L} have acquired molecular characteristics of (cancer) stem cells.

### Discussion

The inventors have shown that constitutive PRLR signaling triggers EMT that in turn transforms luminal (well differentiated) breast cancer cells into cells presenting with i) very aggressive phenotypes and expression profiling typical of undifferentiated/basal-like/mesenchymal cells, ii) hormone-independent proliferation capacities in vitro and in vivo, and iii) stem cell features. Based on these findings, they proposed that the PRLR-I146L genetic variant represents i) a novel mechanism involved in breast cancer progression to aggressive (invasive) stages, and ii) a potential prognostic marker of this evolution. Beyond PRLR-I146L, this model could also apply to any other genetic mutations in the PRLR system leading to constitutive activation thereof. This could involve other gain-of-function mutations of the PRLR (yet to be identified), or to mutation in the promoters of PRL or PRLR genes, leading to over-expression of these proteins (increased transcription or mRNA stability, etc) that could trigger constitutive PRLR signaling.

The identification of PRLR-I146L in breast cancer patients can thus i) predict the progression of this cancer to advanced, hormone-resistant stages, and ii) support the need to down-regulate PRLR signaling in these patients. This could be achieved using specific PRLR antagonists (Bernichtein et al. (2003) J. Biol. Chem. 278:35988-35999; Goffin et al. (2005) Endocr. Rev. 26: 400-422; international application WO03077729) that have been shown to down-regulate the autonomous signaling triggered by PRLR-I146L (Bogorad et al. (2008) Proc. Natl. Acad. Sci. U.S. A 105:14533-14538).

1. An in vitro method of prognosing outcome of hormone-dependent cancer in a subject comprising
obtaining a nucleic acid sample from said subject;
determining, in said nucleic acid sample, a nucleotide sequence of a prolactin receptor (PRLR) gene or a fragment thereof where a constitutively activating mutation might be found; and
detecting a constitutively activating mutation in the nucleotide sequence.

2. The method according to claim 1, wherein said hormone-dependent cancer is breast cancer.

3. The method according to claim 1, wherein the constitutively activating mutation is a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine.

4. The method according to claim 1, wherein the outcome of hormone-dependent cancer in a subject is the progression of the hormone-dependent cancer in said subject.

5. The method according to claim 1, for prognosing progression of the hormone-dependent cancer from a hormone-dependent stage towards a hormone-independent stage.

6. The method according to claim 2 for prognosing progression of the breast cancer towards a hormone receptor-negative breast cancer, a basal-like breast cancer or a triple negative breast cancer.

7. The method according to any claim 2, for prognosing an epithelial to mesenchymal transition of breast cancer cells of said subject.

8. The method according to claim 2, for prognosing progression of the breast cancer towards a metastatic breast cancer.

9. An in vitro method for identifying a subject suffering from hormone-dependent cancer likely to respond to a treatment with an inhibitor of PRLR-triggered signaling cascades comprising
obtaining a nucleic acid sample from said subject;
determining, in said nucleic acid sample, a nucleotide sequence of a prolactin receptor (PRLR) gene or a fragment thereof where a constitutively activating mutation might be found; and
detecting a constitutively activating mutation in the nucleotide sequence.

10. The method according to claim 9, wherein said hormone-dependent cancer is breast cancer.

11-15. (canceled)

16. The method of claim 1, wherein said step of determining includes sequencing the PRLR gene or the fragment thereof.

17. The method of claim 16, wherein said step of sequencing is carried out by direct sequencing of the PRLR gene or the fragment thereof, or by using probes which overlap a position of a single nucleotide polymorphism (SNP) encoding the constitutively activating mutation in the PRLR gene or the fragment thereof.

18. The method of claim 17, wherein said step of direct sequencing includes one or both of sequencing exon 6 of the PRLR gene in both directions using primers matching intronic boundaries, and sequencing exon 5 of the PRLR gene in both directions using primers matching intronic boundaries.

19. The method of claim 1, wherein said steps of determining and/or detecting are carried out by a method selected from the group consisting of: array-based sandwich assays, ligase-based methods, mass-spectroscopy-based methods, PCR-based methods, restriction enzyme-based methods, exonuclease-based methods, dideoxynucleotide-based methods, Genetic Bit Analysis or GBA, Oligonucleotide Ligation Assays (OLAs), and primer-guided nucleotide incorporation procedures.

20. The method of claim 19, wherein said PCR-based methods include performing an ApoI enzymatic digestion of a PCR amplification fragment.

21. The method of claim 19, wherein said restriction enzyme-based methods include restriction of exon 5 of the PRLR gene with restriction enzyme HpyCH4 IV.

22. A method for treating hormone-dependent cancer and/or preventing progression of hormone-dependent cancer to an hormone-independent stage in a subject in need thereof, comprising
administering to said subject an inhibitor of a PRLR-triggered signaling cascade.

23. The method of claim 22, wherein said hormone-dependent cancer is breast cancer.

24. The method of claim 22, wherein the presence of a constitutively activating mutation in the PRLR gene of the subject has been detected in a nucleic acid sample previously obtained from said subject.

25. The method of claim 24, wherein the constitutively activating mutation is a mutation resulting in the expression of a mutant prolactin receptor wherein the isoleucine residue at position 146 is substituted with leucine or a mutation resulting in the expression of a mutant prolactin receptor wherein the isoleucine residue at position 76 is substituted with valine.

26. The method of claim 22, wherein the inhibitor of PRLR-triggered signaling cascades is selected from the group consisting of a PRLR antagonist, a PRLR inverse agonist and an inhibitor of a downstream activated receptor-associated kinase.


SEQUENCE LISTING (40 sequences)

SEQ ID NO: 1 - DNA, 1000 bp, Homo sapiens
ggaggctgaaatccccagacgccggttttctgggctgggctttctgcttactcactccttctccctctttctggattttaccgaccgttcgcgaaacagctttccacacaatggagcttcatgtcctcgtgcaggaagtactcatcgactgatgtggcagactttgctccctgacaaaactaaagaactctcctattcatggaggcgaacactgaggatgctttccacatgaaccctgaagtgaacttctgatacatttcctgcagcaagagaaggcagccaacatgaaggaaaatgtggcatctgcaaccgttttcactctgctactttttctcaacacctgccttctgaatggacagttacctcctggaaaacctgagatctttaaatgtcgttctcccaataaggaaacattcacctgctggtggaggcctgggacagatggaggacttcctaccaattattcactgacttaccacagggaaggagagacactcatgcatgaatgtccagactacataaccggtggccccaactcctgccactttggcaagcagtacacctccatgtggaggacatacatcatgatggtcaatgccactaaccagatgggaagcagtttctcggatgaactttatgtggacgtgacttacatagttcagccagaccctcctttggagctggctgtggaagtaaaacagccagaagacagaaaaccctacctgtggattaaatggtctccacctaccctgattgacttaaaaactggttggttcacgctcctgtatgaaattcgattaaaacccgagaaagcagctgagtgggagatccattttgctgggcagcaaacagagtttaagattctcagcctacatccaggacagaaataccttgtccaggttcgctgcaaaccagaccatggatactggagtgcatggagtccagcgaccttcattcagatacctagtgacttcaccatgaatgatacaaccgtgtggat

SEQ ID NO: 2 - protein, 622 aa, Homo sapiens
  VARIANT (100)..(100): Ile to Val substitution
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 3 - protein, 598 aa, Homo sapiens
  VARIANT (76)..(76): Ile to Val substitution
QLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPRPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 4 - protein, 227 aa, Homo sapiens
  SIGNAL (1)..(28)
  mat_peptide (29)..(227)
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 5 - DNA, 20 bp, Artificial Sequence
  Synthetic primer
aaattgagcctgaagccaac

SEQ ID NO: 6 - DNA, 20 bp, Artificial Sequence
  Synthetic primer
gtggccactgtgcttactga

SEQ ID NO: 7 - DNA, 20 bp, Artificial Sequence
  Synthetic primer
cctgggactccacctacaga

SEQ ID NO: 8 - DNA, 20 bp, Artificial Sequence
  Synthetic primer
tgtgagcaattctgcttgga

SEQ ID NO: 9 - DNA, 20 bp, Artificial Sequence
  Synthetic primer
ggagacaggctggttgttgt

SEQ ID NO: 10 - DNA, 20 bp, Artificial Sequence
  Synthetic primer
ccactgaggcaccattctct

SEQ ID NO: 11 - DNA, 20 bp, Artificial Sequence
  Synthetic primer
cacagtctgctgaggttgga

SEQ ID NO: 12 - DNA, 20 bp, Artificial Sequence
  Synthetic primer
caagctggccttcagatttc

SEQ ID NO: 13 - DNA, 20 bp, Artificial Sequence
  Synthetic primer
actacacgaccatccaggac

SEQ ID NO: 14 - DNA, 20 bp, Artificial Sequence
  Synthetic primer
tctcaaacttggttcggaag

SEQ ID NO: 15 - DNA, 21 bp, Artificial Sequence
  Synthetic primer
tgcctttttgaagaaactcca

SEQ ID NO: 16 - DNA, 20 bp, Artificial Sequence
  Synthetic primer
gtgaggtcaggcttggaaac

SEQ ID NO: 17 - DNA, 20 bp, Artificial Sequence
  Synthetic primer
gccaccatctacctcctcaa

SEQ ID NO: 18 - DNA, 21 bp, Artificial Sequence
  Synthetic primer
cagacagaaggcttttcatcg

SEQ ID NO: 19 - DNA, 20 bp, Artificial Sequence
  Synthetic primer
tccaatggcaagtttgtgac

SEQ ID NO: 20 - DNA, 18 bp, Artificial Sequence
  Synthetic primer
gatgatggggcggttgat

SEQ ID NO: 21 - DNA, 20 bp, Artificial Sequence
  Synthetic primer
gcgaccctaaacacctcaac

SEQ ID NO: 22 - DNA, 20 bp, Artificial Sequence
  Synthetic primer
acagcaagcggtaaaaccag

SEQ ID NO: 23 - DNA, 20 bp, Artificial Sequence
  Synthetic primer
ggctcaactcgcatctcaag

SEQ ID NO: 24 - DNA, 20 bp, Artificial Sequence
  Synthetic primer
tgatttcaaagagggcatgg

SEQ ID NO: 25 - DNA, 20 bp, Artificial Sequence
  Synthetic primer
gtccgcagtcttacgaggag

SEQ ID NO: 26 - DNA, 18 bp, Artificial Sequence
  Synthetic primer
gcagcgtggggatgatct

SEQ ID NO: 27 - DNA, 20 bp, Artificial Sequence
  Synthetic primer
ctctttcctcgtcaggaagc

SEQ ID NO: 28 - DNA, 19 bp, Artificial Sequence
  Synthetic primer
gtcgtagggctgctggaag

SEQ ID NO: 29 - DNA, 19 bp, Artificial Sequence
  Synthetic primer
atttcaacgcctccaaaaa

SEQ ID NO: 30 - DNA, 20 bp, Artificial Sequence
  Synthetic primer
ttgtggtatgacaggcatgg

SEQ ID NO: 31 - DNA, 20 bp, Artificial Sequence
  Synthetic primer
tccttacctgtgggagctgt

SEQ ID NO: 32 - DNA, 20 bp, Artificial Sequence
  Synthetic primer
gcggattttatcaacgatgc

SEQ ID NO: 33 - DNA, 20 bp, Artificial Sequence
  Synthetic primer
tggagtctggtcctgtgagg

SEQ ID NO: 34 - DNA, 20 bp, Artificial Sequence
  Synthetic primer
ggcagctcttcctcctgttt

SEQ ID NO: 35 - DNA, 20 bp, Artificial Sequence
  Synthetic primer
tgtgactgcctgtccctaca

SEQ ID NO: 36 - DNA, 20 bp, Artificial Sequence
  Synthetic primer
gcttgctgcacttctcacac

SEQ ID NO: 37 - DNA, 20 bp, Artificial Sequence
  Synthetic primer
ctgggacagatggaggactt

SEQ ID NO: 38 - DNA, 19 bp, Artificial Sequence
  Synthetic primer
gggccaccggttatgtagt

SEQ ID NO: 39 - DNA, 20 bp, Artificial Sequence
  Synthetic primer
caggtcctggcatcttgtcc

SEQ ID NO: 40 - DNA, 20 bp, Artificial Sequence
  Synthetic primer
ttgctggtcttgccattcct